(19) United States
(12) Patent Application Publication
(10) Pub. No.: US 2026/0031200 A1
(43) Pub. Date: Jan. 29, 2026

(54) ENABLING RISK BASED MONITORING OF A CLINICAL TRIAL

(71) Applicant: ICON Clinical Research Limited, Dublin (IE)

(72) Inventors: Ronan Fox, Dublin (IE); Sean Kelly, Dublin (IE); Thomas O’Leary, Dublin (IE)

(21) Appl. No.: 18/781,601

(22) Filed: Jul. 23, 2024

(51) Int. Cl. G16H 10/20; G16H 50/30

(52) U.S. Cl. CPC G16H 10/20; G16H 50/30

(57) ABSTRACT

According to an embodiment, disclosed is a system comprising a processor configured to define, one or more risk categories for monitoring a risk associated with a clinical trial, wherein the risk categories comprise one or more risk elements; calculate, a first risk profile data of the risk categories based on a risk factor and a weighting assigned to the risk elements; generate, a machine learning (ML) model; train, the ML model; receive, a second risk profile data; analyse, the second risk profile data to identify a pattern based on the first risk profile data using a database; predict, an overall risk score; recommend, one or more of a type of monitoring, a level of monitoring, and the overall risk score; and wherein the ML model comprises a feed-back layer to enable continuous learning and improve the prediction of the overall risk score and monitoring decisions of the clinical trial.

### FIELD OF THE INVENTION

The present disclosure relates to systems and methods for risk based monitoring of a clinical trial. More specifically, the present disclosure relates to systems and methods of risk assessment for clinical trials performed at one or more clinical trial sites to enable early detection and proactive management of clinical trial risks.

### BACKGROUND

“The cost of developing new treatments has always been rising and will continue to do so in the future. Taking into account all the work that needs to be done to gain marketing authorization, the average cost of developing a single new treatment is currently estimated at 2.6 billion USD. Adding up all the research conducted worldwide, the cost of global clinical trials is currently estimated at about 44 billion USD and is expected to reach 69 billion USD by 2026. To curb the ever-rising cost of clinical trials, regulators and technology vendors encourage sponsors to adopt innovative solutions and specifically recommend the use of centralized monitoring (CM) to oversee clinical trials more efficiently.” [Source: How Much Money Can Be Saved by Using Centralized Monitoring in Clinica—xlsmetrics]

“Of all the tasks accomplished by the CRA, Source Data Verification (SDV) can be the most resource-intensive and costly. CRA verification of all CRF/eCRF data, known as 100% SDV, has been the industry norm, even though it is not required by current regulations. Current FDA Draft Guidance states that monitoring should include a mix of centralized monitoring (e.g., through the use of data checks, described below) and on-site monitoring. This mixed approach may reduce monitoring-related time and cost without compromising clinical data quality.” [U.S. Pat. No. 8,706,537B1 granted on Apr. 22, 2014]

“What is needed in the art is the ability to harness the power of computing devices to collect performance data from various sources, analyse the diversity of data presented, and intelligently report analytics that are not capable of being created by a human being or even teams of human beings.” [US11188865B2 granted 30 Nov. 2021]

With the advent of hybrid and decentralized clinical trials, data is sourced from multiple discrete channels. The clinical monitor of the clinical trial must ensure that the data originating from these channels is valid, maintains its integrity, and is accurate. One of the methods used in “traditional” site-focused clinical trials is that of monitoring of the sites through which data is sourced.

Therefore, with patients and the caregivers (both professional and non-professional) now sourcing data directly for a clinical trial, there is a need for an innovative solution to focus on the sources of high risk within the data collection process.

### SUMMARY

The following presents a summary to provide a basic understanding of one or more embodiments described herein. This summary is not intended to identify key or critical elements or delineate any scope of the different embodiments and/or any scope of the claims. The sole purpose of the summary is to present some concepts in a simplified form as a prelude to the more detailed description presented herein.

According to an embodiment, disclosed is a system comprising a processor storing instructions in a non-transitory memory that, when executed, cause the processor to define, one or more risk categories that influence risk associated with monitoring of a clinical trial, wherein the risk categories comprise one or more risk elements; calculate, a first risk profile data of the risk categories based on a risk factor and a weighting assigned to the risk elements; generate, a machine learning model; train, the machine learning model with the first risk profile data; receive, by the machine learning model, a second risk profile data based on the risk categories and the risk elements; analyse, by the machine learning model, the second risk profile data to identify a pattern based on the first risk profile data using a database; predict, by the machine learning model and based on the pattern, an overall risk score for the clinical trial based on a predefined threshold value of the overall risk score; recommend, by the machine learning model based on the risk elements, one or more of a type of monitoring, a level of monitoring, and the overall risk score; update the database with the second risk profile data; and wherein the machine learning model is a self-learning model comprising a feed-back layer that enables the machine learning model to learn from the second risk profile data and improve the prediction of the overall risk score and monitoring decisions to enable risk based monitoring (RBM) of the clinical trial.

According to an embodiment of the system, the risk categories comprise global risk category, local risk category, site specific risk category and patient specific risk category.

According to an embodiment of the system, the risk elements are defined for each of the global risk category, the local risk category, the site specific risk category and the patient specific risk category.

According to an embodiment of the system, the risk elements for the global risk category comprises global risk elements, the risk elements for the local risk category comprises local risk elements, the risk elements for the site specific risk category comprises site specific risk elements, and the risk elements for the patient specific risk category comprises patient specific risk elements.

According to an embodiment of the system, the global risk elements comprises one or more of a therapeutic area, a study phase, a protocol complexity, an interventional risk, and an observational risk.

According to an embodiment of the system, the local risk elements comprises one or more of a geographic area, a socio economic profile, a site maturity profile, and a site experience profile.

According to an embodiment of the system, the site specific risk elements comprises one or more of a site feasibility, a prior history of a site, a site permanency, a site selection, and a site recruitment plan.

According to an embodiment of the system, the patient specific risk elements comprises one or more of a patient recruitment plan, a prior history of a patient, a patient selection criteria, and a patient recruitment forecast.

According to an embodiment of the system, the predefined threshold value of the overall risk score is initially set based on at least one of outcomes of historical clinical trials and predicted by the machine learning model.

According to an embodiment of the system, the second risk profile data comprises planning data sets, wherein the planning data sets further comprises one or more of site recruitment plans, patient recruitment plans, a site forecast information, and a patient forecast information.

According to an embodiment of the system, the processor is further configured to assign a weighting to the risk categories based on a first pattern of outcomes of historical clinical trials and apply the weighting to factor values before generating the overall risk score.

According to an embodiment of the system, the processor is further configured to apply a different weighting to the risk categories at different time periods within the clinical trial.

According to an embodiment of the system, the processor is configured to modify the weighting of the risk elements based on a second pattern of outcomes of historical clinical trials.

According to an embodiment of the system, the processor is further configured to apply a different weighting to the risk elements at different time periods within the clinical trial.

According to an embodiment of the system, the processor is configured to display the overall risk score.

According to an embodiment of the system, the processor is further configured to display a chart showing correlation between the risk categories and the overall risk score.

According to an embodiment of the system, the level of monitoring comprises one or more of on-site monitoring with up to 100% Source Data Verification (SDV), remote monitoring with up to 100% SDV, no monitoring, and variations thereof.

According to an embodiment of the system, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

According to an embodiment of the system, the machine learning model comprises a neural network comprising a non-linear activation function configured to capture a non-linear association with the first risk profile data.

According to an embodiment of the system, the system is further configured for stratifying the type of monitoring into one of low, medium, and high risk categories using the overall risk score.

According to an embodiment of the system, the processor is further configured to perform one or more of a normalization, a standardization, and a stratification of the overall risk score.

According to an embodiment of the system, a high score of the overall risk score indicates a higher probability of monitoring the clinical trial; and wherein a low score of the overall risk score indicates lower probability of monitoring the clinical trial.

According to an embodiment of the system, the processor is further configured to predict the overall risk score using at least one of a logistic regression, a Support Vector Machine (SVM) regression, a convolutional neural network (CNN), a recurrent neural network (RNN), and a long short-term memory model (LSTM).

According to an embodiment of the system, the processor is further configured to predict the overall risk score using the Support Vector Machine (SVM) model.

According to an embodiment of the system, the processor is further configured to predict the overall risk score using an Explainable AI model.

According to an embodiment of the system, the machine learning model comprises an explainable AI algorithm, wherein the explainable AI algorithm is configured to provide reasoning for the prediction of the overall risk score and the monitoring decisions; and build trust parameters for users of the model.

According to an embodiment, disclosed is a method comprising defining, one or more risk categories that influence risk associated with monitoring of a clinical trial, wherein the one or more risk categories comprise one or more risk elements; calculating, a first risk profile data of the one or more risk categories based on a risk factor and a weighting assigned to the one or more risk elements; generating, a machine learning model; training, the machine learning model with the first risk profile data; receiving, by the machine learning model, a second risk profile data based on the one or more risk categories and the one or more risk elements; analysing, by the machine learning model, the second risk profile data to identify a pattern based on the first risk profile data using a database; predicting, by the machine learning model and based on the pattern, an overall risk score for the clinical trial based on a predefined threshold value of the overall risk score; recommending, by the machine learning model based on the risk elements, one or more of a type of monitoring, a level of monitoring, and the overall risk score; updating the database with the second risk profile data; and wherein the machine learning model is a self-learning model comprising a feed-back layer that enables the machine learning model to learn from the second risk profile data and improve the prediction of the overall risk score and monitoring decisions to enable risk based monitoring (RBM) of the clinical trial.

According to an embodiment, disclosed is a non-transitory computer-readable medium having stored thereon instructions executable by a computer system to perform operations comprising, defining, one or more risk categories that influence risk associated with monitoring of a clinical trial, wherein the one or more risk categories comprise one or more risk elements; calculating, a first risk profile data of the one or more risk categories based on a risk factor and a weighting assigned to the one or more risk elements; generating, a machine learning model; training, the machine learning model with the first risk profile data; receiving, by the machine learning model, a second risk profile data based on the one or more risk categories and the one or more risk elements; analysing, by the machine learning model, the second risk profile data to identify a pattern based on the first risk profile data using a database; predicting, by the machine learning model and based on the pattern, an overall risk score for the clinical trial based on a predefined threshold value of the overall risk score; recommending, by the machine learning model based on the one or more risk elements, a type of monitoring, a level of monitoring, and the overall risk score; updating the database with the second risk profile data; and wherein the machine learning model is a self-learning model comprising a feed-back layer that enables the machine learning model to learn from the second risk profile data and improve the prediction of the overall risk score and monitoring decisions to enable risk based monitoring (RBM) of the clinical trial.

According to an embodiment, disclosed is a method comprising, defining, one or more risk categories that influence risk associated with site monitoring of a clinical trial, wherein the one or more risk categories comprise one or more risk elements; calculating, a first site risk profile data of the one or more risk categories based on a risk factor and a weighting assigned to the one or more risk elements; generating, a machine learning model; training, the machine learning model with the first site risk profile data; receiving, by the machine learning model, a second site risk profile data based on the one or more risk categories and the one or more risk elements; analysing, by the machine learning model, the second site risk profile data to identify a pattern based on the first site risk profile data using a database; predicting, by the machine learning model and based on the pattern, a site risk score for the clinical trial based on a predefined threshold value of the site risk score; recommending, by the machine learning model based on the one or more risk elements, a type of monitoring, a level of monitoring, and the site risk score; updating the database with the second site risk profile data; and wherein the machine learning model is a self-learning model comprising a feed-back layer that enables the machine learning model to learn from the second site risk profile data and improve the prediction of the site risk score and monitoring decisions to enable risk based monitoring (RBM) of a site in the clinical trial.

According to an embodiment of the method, the risk categories comprise one or more of a local risk category, a site specific risk category and a patient specific risk category.

According to an embodiment of the method, the risk elements are defined for each of the local risk category, the site specific risk category and the patient specific risk category.

According to an embodiment of the method, the risk elements for the local risk category comprises local risk elements, the risk elements for the site specific risk category comprises site specific risk elements and the risk elements for the patient specific risk category comprises patient specific risk elements.

According to an embodiment of the method, the local risk elements comprises one or more of a geographic area, a socio economic profile, a site maturity profile, and a site experience profile.

According to an embodiment of the method, the site specific risk elements comprises one or more of a site feasibility, a prior history of a site, a site permanency, a site selection, and a site recruitment plan.

According to an embodiment of the method, the patient specific risk elements comprises one or more of a patient recruitment plan, a prior history of a patient, a patient selection criteria, and a patient recruitment forecast.

According to an embodiment of the method, the predefined threshold value of the site risk score is initially set based on outcomes of one or more of historical clinical trials and predicted by the machine learning model.

According to an embodiment of the method, the second site risk profile data comprises planning data sets, wherein the planning data sets further comprises one or more of site recruitment plans, patient recruitment plans, a site forecast information, and a patient forecast information.

According to an embodiment of the method, the method is further configured to assign a weighting to the risk categories based on a first pattern of outcomes of historical clinical trials and apply the weighting to factor values before generating the site risk score.

According to an embodiment of the method, the method is further configured to apply a different weighting to the risk categories at different time periods within the clinical trial.

According to an embodiment of the method, the method is configured to modify the weighting of the risk elements based on a second pattern of outcomes of historical clinical trials.

According to an embodiment of the method, the method is further configured to apply a different weighting to the risk elements at different time periods within the clinical trial.

According to an embodiment of the method, the method is configured to display the site risk score.

According to an embodiment of the method, the method is further configured to display a chart showing correlation between the risk categories and the site risk score.

According to an embodiment of the method, the level of monitoring comprises one or more of on-site monitoring with up to 100% Source Data Verification (SDV), a remote monitoring with up to 100% SDV, no monitoring, and variations thereof.

According to an embodiment of the method, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

According to an embodiment of the method, the machine learning model comprises a neural network comprising a non-linear activation function to capture a non-linear association with the first site risk profile data.

According to an embodiment of the method, the method is further configured for stratifying the type of monitoring into one of low, medium, and high categories using the site risk score.

According to an embodiment of the method, the method is further configured to perform one or more of a normalization, a standardization, and a stratification of the site risk score.

According to an embodiment of the method, higher scores of the site risk score indicates a higher probability of monitoring the clinical trial; and wherein a low score of the site risk score indicates a lower probability of monitoring the clinical trial.

According to an embodiment of the method, the method is further configured to predict the site risk score using at least one of a logistic regression, a Support Vector Machine (SVM) regression, a convolutional neural network (CNN), a recurrent neural network (RNN), and a long short-term memory model (LSTM).

According to an embodiment of the method, the training includes building a site-specific process review plan.

According to an embodiment of the method, the site monitoring includes reporting workflows with process reviews.

According to an embodiment of the method, the method further comprises reporting protocol deviations based on the site monitoring.

According to an embodiment, disclosed is a method comprising, defining, one or more risk categories that influence risk associated with patient monitoring in a clinical trial, wherein the one or more risk categories comprise one or more risk elements; calculating, a first patient risk profile data of the one or more risk categories based on a risk factor and a weighting assigned to the one or more risk elements; generating, a machine learning model; training, the machine learning model with the first patient risk profile data; receiving, by the machine learning model, a second patient risk profile data based on the one or more risk categories and the one or more risk elements; analysing, by the machine learning model, the second patient risk profile data to identify a pattern based on the first patient risk profile data using a database; predicting, by the machine learning model and based on the pattern, a patient risk score for the clinical trial based on a predefined threshold value of the patient risk score; recommending, by the machine learning model based on the one or more risk elements, a type of monitoring, a level of monitoring, and the patient risk score; updating the database with the second patient risk profile data; and wherein the machine learning model is a self-learning model comprising a feed-back layer that enables the machine learning model to learn from the second patient risk profile data and improve the prediction of the patient risk score and monitoring decisions to enable risk based monitoring (RBM) of patient in the clinical trial.

According to an embodiment of the method, the risk categories comprise one or more of a local risk category, a site specific risk category and a patient specific risk category.

According to an embodiment of the method, the risk elements are defined for each of the local risk category, the site specific risk category and the patient specific risk category.

According to an embodiment of the method, the risk elements for the local risk category comprises local risk elements, the risk elements for the site specific risk category comprises site specific risk elements and the risk elements for the patient specific risk category comprises patient specific risk elements.

According to an embodiment of the method, the local risk elements comprises one or more of a geographic area, a socio economic profile, a site maturity profile, and a site experience profile.

According to an embodiment of the method, the site specific risk elements comprises one or more of a site feasibility, a prior history of a site, a site permanency, a site selection, and a site recruitment plan.

According to an embodiment of the method, the patient specific risk elements comprises one or more of a patient recruitment plan, a prior history of a patient, a patient selection criteria, and a patient recruitment forecast.

According to an embodiment of the method, the predefined threshold value of the patient risk score is initially set based on outcomes of at least one of a historical clinical trials and predicted by the machine learning model.

According to an embodiment of the method, the second patient risk profile data comprises planning data sets, wherein the planning data sets further comprise one or more of site recruitment plans, patient recruitment plans, a site forecast information, and a patient forecast information.

According to an embodiment of the method, the method is further configured to assign a weighting to the risk categories based on a first pattern of outcomes of historical clinical trials and apply the weighting to factor values before generating the patient risk score.

According to an embodiment of the method, the method is further configured to apply a different weighting to the risk categories at different time periods within the clinical trial.

According to an embodiment of the method, the method is configured to modify the weighting of the risk elements based on a second pattern of outcomes of historical clinical trials.

According to an embodiment of the method, the method is further configured to apply different weighting to the risk elements at different time periods within the clinical trial.

According to an embodiment of the method, the method is configured to display the patient risk score.

According to an embodiment of the method, the method is further configured to display a chart showing correlation between the risk categories and the patient risk score.

According to an embodiment of the method, the level of monitoring comprises one or more of on-site monitoring with up to 100% Source Data Verification (SDV), remote monitoring with up to 100% SDV, no monitoring, and variations thereof.

According to an embodiment of the method, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

According to an embodiment of the method, the machine learning model comprises a neural network comprising a non-linear activation function to capture a non-linear association with the first patient risk profile data.

According to an embodiment of the method, the method is further configured for stratifying the type of monitoring into one of low, medium, and high categories using the patient risk score.

According to an embodiment of the method, the method is further configured to perform one or more of a normalization, a standardization, and a stratification of the patient risk score.

According to an embodiment of the method, a high score of the patient risk score indicates a higher probability of monitoring the clinical trial; and wherein a low score of the patient risk score indicates a lower probability of monitoring the clinical trial.

According to an embodiment of the method, the method is further configured to predict the patient risk score using at least one of a logistic regression, a Support Vector Machine (SVM) regression, a convolutional neural network (CNN), a recurrent neural network (RNN), and a long short-term memory model (LSTM).

According to an embodiment of the method, the training includes building a patient-specific process review plan.

According to an embodiment of the method, the patient monitoring includes reporting workflows with process reviews.

According to an embodiment of the method, the method further comprises reporting protocol deviations based on the patient monitoring.

According to an embodiment, disclosed is a system comprising a processor storing instructions in a non-transitory memory that, when executed, cause the processor to receive, one of risk categories and risk elements; calculate, a first risk profile data based on a risk factor and a weighting; receive, a second risk profile data from an artificial intelligence engine; analyse, second risk profile data to identify a pattern based on the first risk profile data; predict, an overall risk score for clinical trial based on the pattern; recommend, one or more of a type of monitoring and a level of monitoring based on an overall risk score; update a database with the second risk profile data; and wherein the machine learning model is configured for continual learning from the second risk profile data to improve prediction and monitoring decisions.

According to an embodiment, disclosed is a system comprising a processor storing instructions in a non-transitory memory that, when executed, cause the processor to send one of a one or more risk categories and risk elements to a clinical trial analysis and recommendation system via a client system; send data, wherein the data comprises one or more of a patient data, a site data, a drug molecules, a disease information, and a trial protocol, via the client system; receive, a risk score for a clinical trial based on the risk categories and an overall risk score for the clinical trial; send, a predefined threshold value for the risk score for the clinical trial; receive, a second overall risk score for the clinical trial based on the predefined threshold value of the overall risk score; and receive a recommendation, by the machine learning model based on the risk elements, one or more of a type of monitoring, a level of monitoring, and the overall risk score.

In an embodiment of the system, the machine learning model is configured to learn and predict the overall clinical trial risk score using labelled data using a supervised learning method, wherein the supervised learning method comprises logic using at least one of a decision tree, a logistic regression, a support vector machine, a k-nearest neighbors, a Naïve Bayes, a random forest, a linear regression, a polynomial regression, and a support vector machine for regression.

In an embodiment of the system, the machine learning model is configured to learn from the real-time data and predict the overall clinical trial risk score using an unsupervised learning method, wherein the unsupervised learning method comprises logic using at least one of a k-means clustering, a hierarchical clustering, a hidden Markov model, and an apriori algorithm.

In an embodiment of the system, the machine learning model has a feedback loop, wherein the output from a precious step is fed back to the model in real-time to improve the performance and accuracy of the output of a next step.

In an embodiment of the system, the machine learning model comprises a recurrent neural network model.

In an embodiment of the system, the machine learning model has a feedback loop, wherein the learning is further reinforced with a reward for each true positive of the output of the system.

In an embodiment, the system further comprises a cyber security module wherein the cyber security module comprises an information security management module providing isolation between the communication module and servers.

In an embodiment, the information security management module is operable to, receive data from the communication module, exchange a security key at a start of the communication between the communication module and the server, receive the security key from the server, authenticate an identity of the server by verifying the security key, analyse the security key for a potential cyber security threat, negotiate an encryption key between the communication module and the server, encrypt the data; and transmit the encrypted data to the server when no cyber security threat is detected.

In an embodiment, the information security management module is operable to exchange a security key at a start of the communication between the communication module and the server, receive the security key from the server, authenticate an identity of the server by verifying the security key, analyse the security key for a potential cyber security threat, negotiate an encryption key between the system and the server, receive encrypted data from the server, decrypt the encrypted data, perform an integrity check of the decrypted data and transmit the decrypted data to the communication module when no cyber security threat is detected.

According to an embodiment, disclosed is a system to execute an application for artificial intelligence (AI) enabled clinical trial, the system comprising a server that includes an artificial intelligence (AI) component capable of machine learning, wherein the AI component is configured to store and execute one or more machine learning models for risk based monitoring of a clinical trial; train the one or more machine learning models at least partly based on information received from one or more sites of the clinical trial; test the one or more site machine learning models periodically while collecting more data to determine an accuracy of their one or more machine learning models; and a client device configured to communicate with the server, wherein the client device, in conjunction with the server, is configured to provide a clinical trial risk score, a level or monitoring, and a type of monitoring; collect information from the sites for training of the one or more machine learning models with the risk score, the level or monitoring, and the type of monitoring; receive an update of the sites including the one or more machine learning models; and allow the machine learning model to train on the information received from the sites through the client device.

In an embodiment, disclosed is a method performed by one or more computers of a server system, the method comprising: providing, by the server system, data from one or more clinical trials; receiving, by the server system, a data processing request, wherein the data processing request involves stored research data of a first clinical trial and stored research data of a second clinical trial; generating, by the server system, a combined research data, wherein the data processing request comprises a request to apply a trained machine learning model to data from the first clinical trial and data from the second clinical trial; the method further comprises generating input for the trained machine learning model based on the combined research data, and receiving output that the trained machine learning model provided in response to the generated input; and providing the response determined based on the combined research data comprises providing the output of the trained machine learning model.

In an embodiment, disclosed is a computer-implemented method comprising: determining a current value for each of a plurality of elements of a first site; providing one or more inputs to a machine learning model based on the current value for each of the plurality of the first site, wherein the machine learning model has been trained through a supervised learning process comprising: generating a training data set comprising historical starting values of a particular site element and one or more historical values of additional site elements associated with labels indicating historical subsequent values of the particular site element for a plurality of sites, wherein the particular site element comprises one of: a site feasibility; a site prior history; a site permanency; a site selection; and a site recruitment plan; and using the training data set to train the machine learning model to output predicted values with confidence scores for the particular site element in response to inputs comprising a current value for the particular site element and current values for the additional site elements; determining, based on one or more outputs from the machine learning model in response to the one or more inputs, a predicted value of the particular site element for the first site and a confidence score for the predicted value; identifying a plurality of clinical trials based on the particular site element; generating a probability that the first site will be eligible for each of the plurality of clinical trials at a future time, based on the predicted value of the particular site element for the first site and the confidence score for the predicted value; and providing, a ranked list of recommended clinical trials for the first site based on the probability that the first site will be eligible for each of the plurality of clinical trials at the future time, wherein a given clinical trial of the plurality of clinical trials is only included in the ranked list if a corresponding probability that the first site will be eligible for the given clinical trial at the future time is above a threshold.

In an embodiment, the computer-implemented method comprises: determining a current value for each of a plurality of risk elements of a first patient; providing one or more inputs to a machine learning model based on the current value for each of the plurality of risk elements of the first patient, wherein the machine learning model has been trained through a supervised learning process comprising: generating a training data set comprising historical starting values of a particular patient element and one or more historical values of additional patient elements associated with labels indicating historical subsequent values of the particular patient element for a plurality of patients, wherein the particular patient element comprises: a patient age, a patient location, patient engagement, patient prior experience, and an indication of whether a given medication is being taken accurately; using the training data set to train the machine learning model to output predicted values with confidence scores for the particular patient element in response to inputs comprising a current value for the particular patient element and current values for the additional patient elements; determining, based on one or more outputs from the machine learning model in response to the one or more inputs, a predicted value of the particular patient element for the first patient and a confidence score for the predicted value; identifying a plurality of clinical trials based on the particular patient element; generating a probability that the first patient will be eligible for each of the plurality of clinical trials at a future time, based on the predicted value of the particular patient element for the first patient and the confidence score for the predicted value; and providing, a ranked list of recommended clinical trials for the first patient based on the probability that the first patient will be eligible for each of the plurality of clinical trials at the future time.

In an embodiment, the computer-implemented system to enable risk based monitoring of clinical trials, comprises: at least one data processor; and memory storing instructions which, when executed by the at least one data processor, result in operations comprising: parsing a protocol document designed for a clinical trial, the protocol document comprising information about clinical trial data and events; providing factor data for a plurality of clinical trial sites; and calculating a site burden index for at least one of the sites based on the parsed protocol and the provided factor data, the site burden index providing an AI-derived quantitative measure of impact of the protocol on the site utilizing machine learning algorithms that associate input features to a learning model with one or more numeric ratings describing the site burden and a rule-base comprising fuzzy or crisp rules.

In an embodiment, the system disclosed includes a system for federated learning (FL) utilizing computation capability of edge devices in communication with an FL aggregator to enable risk based monitoring of a clinical trial. The system comprises multiple edge devices of end users, one or more federated learner update repositories, and one or more FL aggregators. Each edge device comprises a federated learner model, configured to send tensors to at least one FL aggregator or federated learner update repository. An FL aggregator includes a federated learner, which may be part of the FL aggregator or a separate module. The FL aggregator and/or federated learner is configured to send tensors to the federated learner update repository. Federated learner update repository comprises a back-end configuration, configured to send model updates to edge devices.

In an embodiment, the system further includes a federated learner update repository, sometimes described as a component of an FL aggregator, comprising a federated learning back-end that collects model updates and evaluations from FLea end users. The FL aggregator can be a high availability system. It organizes models that can be updated based on data from end user edge device updates and performs operations required to make these updates, such as admitting or rejecting proposed updates from end users based on criteria and metadata sent by end user. The FL aggregator combines admissible end user updates into an overall update and redistributes the updated model to edge devices.

In an embodiment, the method further includes training a deep neural network (DNN) model on the decentralized data from the sites of the clinical trial, wherein DNN model training comprises: partitioning dataset into several subsets, and assigning each subset to a different device; training a local DNN model by each device on its dataset and sending the model updates to a central server, wherein a stochastic gradient descent (SGD) optimizer is used to train the local models; aggregating the updates by the central server using a weighted average to create a global model, wherein a weighted averaging method is used to aggregate the updates; and sending back the global model to the devices for the next round of training.

In an embodiment, the method further includes generating additional samples to increase the diversity of the dataset using a Generative adversarial network (GAN), wherein the generator network is trained on the aggregated data from the Federated Learning (FL) process and the discriminator network is trained on a small subset of the data that is known to be clean, wherein a Wasserstein GAN (WGAN) technique is used to train the GAN and a gradient penalty regularization is used to improve the stability of the WGAN, wherein the generator network is trained to generate samples that are difficult for the discriminator to distinguish from real samples.

In an embodiment, the method further includes detecting anomalies in network traffic using the trained DNN model, wherein the model is trained to identify patterns that are indicative of attacks upon classifying traffic flows as either benign or malicious using a binary classification approach, wherein the generator network is used to generate additional samples to increase the diversity of the dataset for preventing attackers to bypass the model, wherein detecting and mitigating cyber security threats using a deep neural network (DNN) trained on federated datasets and generative adversarial networks (GAN) comprising establishing a federated learning network of multiple devices, each with its own dataset of network traffic flows and their corresponding labels; defining the architecture of the DNN, including the number of hidden layers and the number of neurons in each layer; selecting appropriate activation functions and optimization techniques for the DNN; training the DNN on the federated datasets using appropriate hyperparameters, including a learning rate and a regularization parameter; using a GAN to generate synthetic network traffic flows for the DNN to train on, wherein the network traffic flows in the dataset are encoded using a one-hot encoding scheme to represent the different features of each flow; evaluating the accuracy and robustness of the DNN using appropriate metrics, including the confusion matrix, the receiver operating characteristic (ROC) curve, and the area under the curve (AUC); detecting cyber security threats in real-time using the trained DNN, such as malware, network intrusion, and data exfiltration; and mitigating cyber security threats by taking appropriate actions based on the detection results, such as blocking suspicious network traffic or isolating infected devices.

The system helps the trial organizers and sponsors in providing preventive strategies based on the risks identified and therefore reduces the cost of interventions by targeted interventions in high risk sites as against performing unnecessary interventions in all sites. Furthermore, the system helps trial monitors in managing low risk sites and patients in terms of costs and time of the caregivers.

### BRIEF DESCRIPTION OF THE FIGURES

These and other aspects of the present invention will now be described in more detail, with reference to the appended drawings showing exemplary embodiments of the present invention, in which:

FIG. 1 is a diagram illustrating a clinical trial network including the main hardware components, according to one or more embodiments.

FIG. 2 shows a flow diagram for a system for enabling risk based monitoring (RBM) in a clinical trial according to one or more embodiments.

FIG. 3 illustrates features of an activity review tool system according to embodiments of the present invention.

FIG. 4 illustrates exemplary features according to embodiments of the present invention.

FIG. 5 illustrates some of the categories of risk profiling along with some examples of the risk elements that may apply to each category.

FIG. 6 is a plot generated by a prediction model of the network, indicating a suggested starting risk profile across global locations based on global and local risk attributes according to one or more embodiments.

FIG. 7 is a plot showing temporal risk according to one or more embodiments.

FIG. 8 shows use of the AI unit of the system for risk assessment, according to one or more embodiments.

FIG. 9A shows a structure of the neural network/machine learning model with a feedback loop.

FIG. 9B shows a structure of the neural network/machine learning model with reinforcement learning.

FIG. 9C shows an example block diagram for detecting overall risk score for a clinical trial using a machine learning model.

FIG. 10A shows a block diagram of the cyber security module in view of the system and server.

FIG. 10B shows an embodiment of the cyber security module.

FIG. 10C shows another embodiment of the cyber security module.

FIG. 11 shows a flow diagram for a method for enabling risk based monitoring (RBM) in a clinical trial according to one or more embodiments.

FIG. 12 shows a flow diagram for a non-transitory computer-readable medium for enabling risk based monitoring (RBM) in a clinical trial according to one or more embodiments.

FIG. 13 shows a flow diagram for a method for enabling risk based monitoring (RBM) of a site in a clinical trial according to one or more embodiments.

FIG. 14 shows a flow diagram for a method for enabling risk based monitoring (RBM) of a patient in a clinical trial according to one or more embodiments.

### DETAILED DESCRIPTION

For simplicity and clarity of illustration, the figures illustrate the general manner of construction. The description and figures may omit the descriptions and details of well-known features and techniques to avoid unnecessarily obscuring the present disclosure. The figures exaggerate the dimensions of some of the elements relative to other elements to help improve understanding of embodiments of the present disclosure. The same reference numeral in different figures denotes the same element.

Although the detailed description herein contains many specifics for the purpose of illustration, a person of ordinary skill in the art will appreciate that many variations and alterations to the details are considered to be included herein.

Accordingly, the embodiments herein are without any loss of generality to, and without imposing limitations upon, any claims set forth. The terminology used herein is for the purpose of describing particular embodiments only and is not limiting. Unless defined otherwise, all technical and scientific terms used herein have the same meaning as commonly understood by one with ordinary skill in the art to which this disclosure belongs. The following terms and phrases, unless otherwise indicated, shall be understood to have the following meanings.

As used herein, the articles “a” and “an” used herein refer to one or to more than one (i.e., to at least one) of the grammatical object of the article. By way of example, “an element” means one element or more than one element. Moreover, usage of articles “a” and “an” in the subject specification and annexed drawings construe to mean “one or more” unless specified otherwise or clear from context to mean a singular form.

As used herein, the terms “example” and/or “exemplary” mean serving as an example, instance, or illustration. For the avoidance of doubt, such examples do not limit the herein described subject matter. In addition, any aspect or design described herein as an “example” and/or “exemplary” is not necessarily preferred or advantageous over other aspects or designs, nor does it preclude equivalent exemplary structures and techniques known to those of ordinary skill in the art.

As used herein, the terms “first,” “second,” “third,” and the like in the description and in the claims, if any, distinguish between similar elements and do not necessarily describe a particular sequence or chronological order. The terms are interchangeable under appropriate circumstances such that the embodiments herein are, for example, capable of operation in sequences other than those illustrated or otherwise described herein. Furthermore, the terms “include,” “have,” and any variations thereof, cover a non-exclusive inclusion such that a process, method, system, article, device, or apparatus that comprises a list of elements is not necessarily limiting to those elements, but may include other elements not expressly listed or inherent to such process, method, system, article, device, or apparatus.

As used herein, the terms “left,” “right,” “front,” “back,” “top,” “bottom,” “over,” “under” and the like in the description and in the claims, if any, are for descriptive purposes and not necessarily for describing permanent relative positions. The terms so used are interchangeable under appropriate circumstances such that the embodiments of the apparatus, methods, and/or articles of manufacture described herein are, for example, capable of operation in other orientations than those illustrated or otherwise described herein.

No element act, or instruction used herein is critical or essential unless explicitly described as such. Furthermore, the term “set” includes items (e.g., related items, unrelated items, a combination of related items and unrelated items, etc.) and may be interchangeable with “one or more.” Where only one item is intended, the term “one” or similar language is used. Also, the terms “has,” “have,” “having,” or the like are open-ended terms. Further, the phrase “based on” means “based, at least in part, on” unless explicitly stated otherwise.

As used herein, the terms “system,” “device,” “unit,” and/or “module” refer to a different component, component portion, or component of the various levels of the order. However, other expressions that achieve the same purpose may replace the terms.

As used herein, the terms “couple,” “coupled,” “couples,” “coupling,” and the like refer to connecting two or more elements mechanically, electrically, and/or otherwise. Two or more electrical elements may be electrically coupled together, but not mechanically or otherwise coupled together. Coupling may be for any length of time, e.g., permanent, or semi-permanent or only for an instant. “Electrical coupling” includes electrical coupling of all types. The absence of the word “removably,” “removable,” and the like, near the word “coupled” and the like does not mean that the coupling, etc. in question is or is not removable.

As used herein, the term “or” means an inclusive “or” rather than an exclusive “or.” That is, unless specified otherwise, or clear from context, “X employs A or B” means any of the natural inclusive permutations. That is, if X employs A; X employs B; or X employs both A and B, then “X employs A or B” is satisfied under any of the foregoing instances.

As used herein, two or more elements or modules are “integral” or “integrated” if they operate functionally together. Two or more elements are “non-integral” if each element can operate functionally independently.

As used herein, the term “real-time” refers to operations conducted as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

As used herein, the term “approximately” can mean within a specified or unspecified range of the specified or unspecified stated value. In some embodiments, “approximately” can mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

As used herein the term “component” refers to a distinct and identifiable part, element, or unit within a larger system, structure, or entity. It is a building block that serves a specific function or purpose within a more complex whole. Components are often designed to be modular and interchangeable, allowing them to be combined or replaced in various configurations to create or modify systems. Components may be a combination of mechanical, electrical, hardware, firmware, software and/or other engineering elements.

Digital electronic circuitry, or computer software, firmware, or hardware, including the structures disclosed in this specification and their structural equivalents, or in combinations of one or more of them may realize the implementations and all of the functional operations described in this specification. Implementations may be as one or more computer program products i.e., one or more modules of computer program instructions encoded on a computer-readable medium for execution by, or to control the operation of, data processing apparatus. The computer-readable medium may be a machine-readable storage device, a machine-readable storage substrate, a memory device, a composition of matter affecting a machine-readable propagated signal, or a combination of one or more of them. The term “computing system” encompasses all apparatus, devices, and machines for processing data, including by way of example, a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal (e.g., a machine-generated electrical, optical, or electromagnetic signal) that encodes information for transmission to a suitable receiver apparatus.

The actual specialized control hardware or software code used to implement these systems and/or methods is not limiting to the implementations. Thus, any software and any hardware can implement the systems and/or methods based on the description herein without reference to specific software code.

A computer program (also known as a program, software, software application, script, or code) is written in any appropriate form of programming language, including compiled or interpreted languages. Any appropriate form, including a standalone program or a module, component, subroutine, or other unit suitable for use in a computing environment may deploy it. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may execute on one computer or on multiple computers that are located at one site or distributed across multiple sites and interconnected by a communication network.

One or more programmable processors, executing one or more computer programs to perform functions by operating on input data and generating output, perform the processes and logic flows described in this specification. The processes and logic flows may also be performed by, and apparatus may also be implemented as, special purpose logic circuitry, for example, without limitation, a Field Programmable Gate Array (FPGA), an Application Specific Integrated Circuit (ASIC), Application Specific Standard Products (ASSPs), System-On-a-Chip (SOC) systems, Complex Programmable Logic Devices (CPLDs), etc.

Processors suitable for the execution of a computer program include, by way of example, both general and special purpose microprocessors, and any one or more processors of any appropriate kind of digital computer. A processor will receive instructions and data from a read-only memory or a random-access memory or both. Elements of a computer can include a processor for performing instructions and one or more memory devices for storing instructions and data. A computer will also include, or is operatively coupled to receive data, transfer data or both, to/from one or more mass storage devices for storing data e.g., magnetic disks, magneto optical disks, optical disks, or solid-state disks. However, a computer need not have such devices. Moreover, another device, e.g., a mobile telephone, a personal digital assistant (PDA), a mobile audio player, a Global Positioning System (GPS) receiver, etc. may embed a computer. Computer-readable media suitable for storing computer program instructions and data include all forms of non-volatile memory, media and memory devices, including, by way of example, semiconductor memory devices (e.g., Erasable Programmable Read-Only Memory (EPROM), Electronically Erasable Programmable Read-Only Memory (EEPROM), and flash memory devices), magnetic disks (e.g., internal hard disks or removable disks), magneto optical disks (e.g. Compact Disc Read-Only Memory (CD ROM) disks, Digital Versatile Disk-Read-Only Memory (DVD-ROM) disks) and solid-state disks. Special purpose logic circuitry may supplement or incorporate the processor and the memory.

To provide for interaction with a user, a computer may have a display device, e.g., a Cathode Ray Tube (CRT) or Liquid Crystal Display (LCD) monitor, for displaying information to the user, and a keyboard and a pointing device, e.g., a mouse or a trackball, by which the user may provide input to the computer. Other kinds of devices provide for interaction with a user as well. For example, feedback to the user may be any appropriate form of sensory feedback, e.g., visual feedback, auditory feedback, or tactile feedback; and a computer may receive input from the user in any appropriate form, including acoustic, speech, or tactile input.

A computing system that includes a back-end component, e.g., a data server, or that includes a middleware component, e.g., an application server, or that includes a front-end component, e.g., a client computer having a graphical user interface or a Web browser through which a user may interact with an implementation, or any appropriate combination of one or more such back-end, middleware, or front-end components, may realize implementations described herein. Any appropriate form or medium of digital data communication, e.g., a communication network may interconnect the components of the system. Examples of communication networks include a Local AreaNetwork (LAN) and a Wide AreaNetwork (WAN), e.g., Intranet and Internet.

The computing system may include clients and servers. A client and server are remote from each other and typically interact through a communication network. The relationship of the client and server arises by virtue of computer programs running on the respective computers and having a client-server relationship with each other.

Embodiments of the present invention may comprise or utilize a special purpose or general purpose computer including computer hardware. Embodiments within the scope of the present invention may also include physical and other computer-readable media for carrying or storing computer-executable instructions and/or data structures. Such computer-readable media can be any media accessible by a general purpose or special purpose computer system. Computer-readable media that store computer-executable instructions are physical storage media. Computer-readable media that carry computer-executable instructions are transmission media. Thus, by way of example and not limitation, embodiments of the invention can comprise at least two distinct kinds of computer-readable media: physical computer-readable storage media and transmission computer-readable media.

Although the present embodiments described herein are with reference to specific example embodiments it will be evident that various modifications and changes may be made to these embodiments without departing from the broader spirit and scope of the various embodiments. For example, hardware circuitry (e.g., Complementary Metal Oxide Semiconductor (CMOS) based logic circuitry), firmware, software (e.g., embodied in a non-transitory machine-readable medium), or any combination of hardware, firmware, and software may enable and operate the various devices, units, and modules described herein. For example, transistors, logic gates, and electrical circuits (e.g., Application Specific Integrated Circuit (ASIC) and/or Digital Signal Processor (DSP) circuit) may embody the various electrical structures and methods.

In addition, a non-transitory machine-readable medium and/or a system may embody the various operations, processes, and methods disclosed herein. Accordingly, the specification and drawings are illustrative rather than restrictive.

Physical computer-readable storage media includes RAM, ROM, EEPROM, CD-ROM or other optical disk storage (such as CDs, DVDs, etc.), magnetic disk storage or other magnetic storage devices, solid-state disks or any other medium. They store desired program code in the form of computer-executable instructions or data structures which can be accessed by a general purpose or special purpose computer.

As used herein, the term “network” refers to one or more data links that enable the transport of electronic data between computer systems and/or modules and/or other electronic devices. When a network or another communications connection (either hardwired, wireless, or a combination of hardwired or wireless) transfers or provides information to a computer, the computer properly views the connection as a transmission medium. A general purpose or special purpose computer access transmission media that can include a network and/or data links which carry desired program code in the form of computer-executable instructions or data structures. The scope of computer-readable media includes combinations of the above, that enable the transport of electronic data between computer systems and/or modules and/or other electronic devices. The term network may include the Internet, a local area network, a wide area network, or combinations thereof. The network may include one or more networks or communication systems, such as the Internet, the telephone system, satellite networks, cable television networks, and various other private and public networks. In addition, the connections may include wired connections (such as wires, cables, fiber optic lines, etc.), wireless connections, or combinations thereof. Furthermore, although not shown, other computers, systems, devices, and networks may also be connected to the network. Network refers to any set of devices or subsystems connected by links joining (directly or indirectly) a set of terminal nodes sharing resources located on or provided by network nodes.

The computers use common communication protocols over digital interconnections to communicate with each other. For example, subsystems may comprise the cloud. Cloud refers to servers that are accessed over the Internet, and the software and databases that run on those servers.

Further, upon reaching various computer system components, program code in the form of computer-executable instructions or data structures can be transferred automatically from transmission computer-readable media to physical computer-readable storage media (or vice versa). For example, computer-executable instructions or data structures received over a network or data link can be buffered in RAM within a Network Interface Module (NIC), and then eventually transferred to computer system RAM and/or to less volatile computer-readable physical storage media at a computer system. Thus, computer system components that also (or even primarily) utilize transmission media may include computer-readable physical storage media.

Computer-executable instructions comprise, for example, instructions and data which cause a general purpose computer, special purpose computer, or special purpose processing device to perform a certain function or group of functions. The computer-executable instructions may be, for example, binary, intermediate format instructions such as assembly language, or even source code. Although the subject matter herein described is in a language specific to structural features and/or methodological acts, the described features or acts described do not limit the subject matter defined in the claims. Rather, the herein described features and acts are example forms of implementing the claims.

While this specification contains many specifics, these do not construe as limitations on the scope of the disclosure or of the claims, but as descriptions of features specific to particular implementations. A single implementation may implement certain features described in this specification in the context of separate implementations. Conversely, multiple implementations separately or in any suitable sub-combination may implement various features described herein in the context of a single implementation. Moreover, although features described herein as acting in certain combinations and even initially claimed as such, one or more features from a claimed combination may in some cases be excised from the combination, and the claimed combination may be directed to a sub-combination or variation of a sub-combination.

Similarly, while operations depicted herein in the drawings in a particular order to achieve desired results, this should not be understood as requiring that such operations be performed in the particular order shown or in sequential order or that all illustrated operations be performed, to achieve desirable results. In certain circumstances, multitasking and parallel processing may be advantageous. Moreover, the separation of various system components in the implementations should not be understood as requiring such separation in all implementations, and it should be understood that the described program components and systems may be integrated together in a single software product or packaged into multiple software products.

Even though particular combinations of features are recited in the claims and/or disclosed in the specification, these combinations are not intended to limit the disclosure of possible implementations. Other implementations are within the scope of the claims. For example, the actions recited in the claims may be performed in a different order and still achieve desirable results. In fact, many of these features may be combined in ways not specifically recited in the claims and/or disclosed in the specification. Although each dependent claim may directly depend on only one claim, the disclosure of possible implementations includes each dependent claim in combination with every other claim in the claim set.

Further, a computer system including one or more processors and computer-readable media such as computer memory may practice the methods. In particular, one or more processors execute computer-executable instructions, stored in the computer memory, to perform various functions such as the acts recited in the embodiments.

Those skilled in the art will appreciate that the invention may be practiced in network computing environments with many types of computer system configurations including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, etc. Distributed system environments where local and remote computer systems, which are linked (either by hardwired data links, wireless data links, or by a combination of hardwired and wireless data links) through a network, both perform tasks may also practice the invention. In a distributed system environment, program modules may be located in both local and remote memory storage devices.

The following terms and phrases, unless otherwise indicated, shall be understood to have the following meanings.

As used herein, the term “Unauthorized access” is when someone gains access to a website, program, server, service, or other system using someone else's account or other methods. For example, if someone kept guessing a password or usemame for an account that was not theirs until they gained access, it is considered unauthorized access.

As used herein, the term “IoT” stands for Internet of Things which describes the network of physical objects “things” or objects embedded with sensors, software, and other technologies for the purpose of connecting and exchanging data with other devices and systems over the internet.

As used herein “Machine learning” refers to algorithms that give a computer the ability to learn without explicit programming, including algorithms that learn from and make predictions about data. Machine learning techniques include, but are not limited to, support vector machine, artificial neural network (ANN) (also referred to herein as a “neural net”), deep learning neural network, logistic regression, discriminant analysis, random forest, linear regression, rules-based machine learning, Naïve Bayes, nearest neighbor, decision tree, decision tree learning, and hidden Markov, etc. For the purposes of clarity, part of a machine learning process can use algorithms such as linear regression or logistic regression. However, using linear regression or another algorithm as part of a machine learning process is distinct from performing a statistical analysis such as regression with a spreadsheet program. The machine learning process can continually learn and adjust the classifier as new data becomes available and does not rely on explicit or rules-based programming. The ANN may be featured with a feedback loop to adjust the system output dynamically as it learns from the new data as it becomes available. In machine learning, backpropagation and feedback loops are used to train the Artificial Intelligence/Machine Learning (AI/ML) model improving the model's accuracy and performance over time. Statistical modeling relies on finding relationships between variables (e.g., mathematical equations) to predict an outcome.

As used herein, the term “Data mining” is a process used to turn raw data into useful information. It is the process of analysing large datasets to uncover hidden patterns, relationships, and insights that can be useful for decision-making and prediction.

As used herein, the term “Data acquisition” is the process of sampling signals that measure real world physical conditions and converting the resulting samples into digital numeric values that a computer manipulates. Data acquisition systems typically convert analog waveforms into digital values for processing. The components of data acquisition systems include sensors to convert physical parameters to electrical signals, signal conditioning circuitry to convert sensor signals into a form that can be converted to digital values, and analog-to-digital converters to convert conditioned sensor signals to digital values. Stand-alone data acquisition systems are often called data loggers.

As used herein, the term “Dashboard” is a type of interface that visualizes particular Key Performance Indicators (KPIs) or Key Result Indicators (KRIs) for a specific goal or process. It is based on data visualization and infographics.

As used herein, a “Database” is a collection of organized information so that it can be easily accessed, managed, and updated. Computer databases typically contain aggregations of data records or files.

As used herein, the term “Data set” (or “Dataset”) is a collection of data. In the case of tabular data, a data set corresponds to one or more database tables, where every column of a table represents a particular variable, and each row corresponds to a given record of the data set in question. The data set lists values for each of the variables, such as height and weight of an object, for each member of the data set. Each value is known as a datum. Data sets can also consist of a collection of documents or files.

As used herein, a “sensor” is a device that detects and measures physical properties from the surrounding environment and converts this information into electrical or digital signals for further processing. Sensors play a crucial role in collecting data for various applications across industries. Sensors may be made of electronic, mechanical, chemical, or other engineering components. Examples include sensors to measure temperature, pressure, humidity, proximity, light, acceleration, orientation etc.

The term “communication module” or “communication system” as used herein refers to a system which enables the information exchange between two points. The process of transmission and reception of information is called communication. The elements of communication include but are not limited to a transmitter of information, channel or medium of communication and a receiver of information.

The term “communication” as used herein refers to the transmission of information and/or data from one point to another. Communication may be by means of electromagnetic waves. Communication is also a flow of information from one point, known as the source, to another, the receiver. Communication comprises one of the following: transmitting data, instructions, information or a combination of data, instructions, and information. Communication happens between any two communication systems or communicating units.

The term “protocol” as used herein refers to a procedure required to initiate and maintain communication; a formal set of conventions governing the format and relative timing of message exchange between two communications terminals; a set of conventions that govern the interactions of processes, devices, and other components within a system; a set of signaling rules used to convey information or commands between boards connected to the bus; a set of signaling rules used to convey information between agents; a set of semantic and syntactic rules that determine the behaviour of entities that interact; a set of rules and formats (semantic and syntactic) that determines the communication behaviour of simulation applications; a set of conventions or rules that govern the interactions of processes or applications between communications terminals; a formal set of conventions governing the format and relative timing of message exchange between communications terminals; a set of semantic and syntactic rules that determine the behaviour of functional units in achieving meaningful communication; a set of semantic and syntactic rules for exchanging information.

The term “communication protocol” as used herein refers to standardized communication between any two systems. An example communication protocol is a Dedicated short-range communications (DSRC) protocol. The DSRC protocol uses a specific frequency band (e.g., 5.9 GHz (Gigahertz)) and specific message formats (such as the Basic Safety Message, Signal Phase and Timing, and Roadside Alert) to enable communications between vehicles and infrastructure components, such as traffic signals and roadside sensors. DSRC is a standardized protocol, and its specifications are maintained by various organizations, including the Institute of Electrical and Electronics Engineers (IEEE) and Society of Automotive Engineers (SAE) International.

The term “bidirectional communication” as used herein refers to an exchange of data between two components. In an example, the first component can be a vehicle and the second component can be an infrastructure that is enabled by a system of hardware, software, and firmware.

The term “in communication with” as used herein, refers to any coupling, connection, or interaction using signals to exchange information, message, instruction, command, and/or data, using any system, hardware, software, protocol, or format regardless of whether the exchange occurs wirelessly or over a wired connection.

The terms “non-transitory computer-readable medium” and “computer-readable medium” include a single medium or multiple media such as a centralized or distributed database, and/or associated caches and servers that store one or more sets of instructions. Further, the terms “non-transitory computer-readable medium” and “computer-readable medium” include any tangible medium that is capable of storing, encoding, or carrying a set of instructions for execution by a processor that, for example, when executed, cause a system to perform any one or more of the methods or operations disclosed herein. As used herein, the term “computer-readable medium” is expressly defined to include any type of computer-readable storage device and/or storage disk and to exclude propagating signals.

The term “application server” refers to a server that hosts applications or software that delivers a business application through a communication protocol. An application server framework is a service layer model. It includes software components available to a software developer through an application programming interface. It is system software that resides between the operating system (OS) on one side, the external resources such as a database management system (DBMS), communications and Internet services on another side, and the users' applications on the third side.

The term “rule-based system” as used herein comprises a set of facts of a scenario and a set of rules for how to deal with the set of facts comprising if and then statements, wherein the scenario is predefined in a system.

The term “cyber security” as used herein refers to application of technologies, processes, and controls to protect systems, networks, programs, devices, and data from cyber-attacks.

The term “cyber security module” as used herein refers to a module comprising application of technologies, processes, and controls to protect systems, networks, programs, devices and data from cyber-attacks and threats. It aims to reduce the risk of cyber-attacks and protect against the unauthorized exploitation of systems, networks, and technologies. It includes, but is not limited to, critical infrastructure security, application security, network security, cloud security, Internet of Things (IoT) security.

The term “encrypt” used herein refers to securing digital data using one or more mathematical techniques, along with a password or “key” used to decrypt the information. It refers to converting information or data into a code, especially to prevent unauthorized access. It may also refer to concealing information or data by converting it into a code. It may also be referred to as cipher, code, encipher, encode. A simple example is representing alphabets with numbers—say, ‘A’ is ‘01’, ‘B’ is ‘02’, and so on. For example, a message like “HELLO” will be encrypted as “0805121215,” and this value will be transmitted over the network to the recipient(s).

The term “decrypt” used herein refers to the process of converting an encrypted message back to its original format. It is generally a reverse process of encryption. It decodes the encrypted information so that only an authorized user can decrypt the data because decryption requires a secret key or password. This term could be used to describe a method of unencrypting the data manually or unencrypting the data using the proper codes or keys.

The term “cyber security threat” used herein refers to any possible malicious attack that seeks to unlawfully access data, disrupt digital operations, or damage information. A malicious act includes but is not limited to damaging data, stealing data, or disrupting digital life in general. Cyber threats include, but are not limited to, malware, spyware, phishing attacks, ransomware, zero-day exploits, trojans, advanced persistent threats, wiper attacks, data manipulation, data destruction, rogue software, malvertising, unpatched software, computer viruses, man-in-the-middle attacks, data breaches, Denial of Service (DoS) attacks, and other attack vectors.

The term “hash value” used herein can be thought of as fingerprints for files. The contents of a file are processed through a cryptographic algorithm, and a unique numerical value, the hash value, is produced that identifies the contents of the file. If the contents are modified in any way, the value of the hash will also change significantly. Example algorithms used to produce hash values: the Message Digest-5 (MD5) algorithm and Secure Hash Algorithm-1 (SHA1).

The term “integrity check” as used herein refers to the checking for accuracy and consistency of system related files, data, etc. It may be performed using checking tools that can detect whether any critical system files have been changed, thus enabling the system administrator to look for unauthorized alteration of the system. For example, data integrity corresponds to the quality of data in the databases and to the level by which users examine data quality, integrity, and reliability. Data integrity checks verify that the data in the database is accurate, and functions as expected within a given application.

The term “alarm” as used herein refers to a trigger when a component in a system or the system fails or does not perform as expected. The system may enter an alarm state when a certain event occurs. An alarm indication signal is a visual signal to indicate the alarm state. For example, when a cyber security threat is detected, a system administrator may be alerted via sound alarm, a message, a glowing LED, a pop-up window, etc. Alarm indication signal may be reported downstream from a detecting device, to prevent adverse situations or cascading effects.

The term “in communication with” as used herein, refers to any coupling, connection, or interaction using electrical signals to exchange information or data, using any system, hardware, software, protocol, or format, regardless of whether the exchange occurs wirelessly or over a wired connection.

As used herein, the term “cryptographic protocol” is also known as security protocol or encryption protocol. It is an abstract or concrete protocol that performs a security-related function and applies cryptographic methods often as sequences of cryptographic primitives. A protocol describes how the algorithms should be used. A sufficiently detailed protocol includes details about data structures and representations, at which point it can be used to implement multiple, interoperable versions of a program. Cryptographic protocols are widely used for secure application-level data transport. A cryptographic protocol usually incorporates at least some of these aspects: key agreement or establishment, entity authentication, symmetric encryption, and message authentication material construction, secured application-level data transport, non-repudiation methods, secret sharing methods, and secure multi-party computation. Hashing algorithms may be used to verify the integrity of data. Secure Socket Layer (SSL) and Transport Layer Security (TLS), the successor to SSL, are cryptographic protocols that may be used by networking switches to secure data communications over a network.

The embodiments described herein can be directed to one or more of a system, a method, an apparatus, and/or a computer program product at any possible technical detail level of integration. The computer program product can include a computer-readable storage medium (or media) having computer-readable program instructions thereon for causing a processor to carry out aspects of the one or more embodiments described herein.

The flowcharts and block diagrams in the figures illustrate the architecture, functionality and/or operation of possible implementations of systems, computer-implementable methods and /or computer program products according to one or more embodiments described herein. In this regard, each block in the flowchart or block diagrams can represent a module, segment and/or portion of instructions, which comprises one or more executable instructions for implementing the specified logical function(s). In one or more alternative implementations, the functions noted in the blocks can occur out of the order noted in the Figures. For example, two blocks shown in succession can be executed substantially concurrently, and/or the blocks can sometimes be executed in the reverse order, depending upon the functionality involved. It will also be noted that each block of the block diagrams and/or flowchart illustration, and/or combinations of blocks in the block diagrams and/or flowchart illustration, can be implemented by special purpose hardware-based systems that can perform the specified functions and/or acts and/or carry out one or more combinations of special purpose hardware and/or computer instructions.

As used in this application, the terms “component,” “system,” “platform,” “interface,” and/or the like, can refer to and/or can include a computer-related entity or an entity related to an operational machine with one or more specific functionalities. The entities described herein can be either hardware, a combination of hardware and software, software, or software in execution. For example, a component can be, but is not limited to being, a process running on a processor, a processor, an object, an executable, a thread of execution, a program and/or a computer. By way of illustration, both an application running on a server and the server can be a component. One or more components can reside within a process and/or thread of execution and a component can be localized on one computer and/or distributed between two or more computers. In another example, respective components can execute from various computer-readable media having various data structures stored thereon. The components can communicate via local and/or remote processes such as in accordance with a signal having one or more data packets (e.g., data from one component interacting with another component in a local system, distributed system and/or across a network such as the Internet with other systems via the signal). As another example, a component can be an apparatus with specific functionality provided by mechanical parts operated by electric or electronic circuitry, which is operated by a software and/or firmware application executed by a processor. In such a case, the processor can be internal and/or external to the apparatus and can execute at least a part of the software and/or firmware application. As yet another example, a component can be an apparatus that provides specific functionality through electronic components without mechanical parts, where the electronic components can include a processor and/or other means to execute software and/or firmware that confers at least in part the functionality of the electronic components. In an aspect, a component can emulate an electronic component via a virtual machine, e.g., within a cloud computing system.

The embodiments described herein include mere examples of systems and computer-implemented methods. It is, of course, not possible to describe every conceivable combination of components and/or computer-implemented methods for purposes of describing the one or more embodiments, but one of ordinary skill in the art can recognize that many further combinations and/or permutations of the one or more embodiments are possible. Furthermore, to the extent that the terms “includes,” “has,” “possesses,” and the like are used in the detailed description, claims, appendices and/or drawings such terms are intended to be inclusive in a manner similar to the term “comprising” as “comprising” is interpreted when employed as a transitional word in a claim.

The descriptions of the one or more embodiments are for purposes of illustration but are not exhaustive or limiting to the embodiments described herein. Many modifications and variations will be apparent to those of ordinary skill in the art without departing from the scope and spirit of the described embodiments. The terminology used herein best explains the principles of the embodiments, the practical application and/or technical improvement over technologies found in the marketplace, and/or to enable others of ordinary skill in the art to understand the embodiments described herein.

As referred herein, “clinical trial” is a research study in which one or more human subjects are prospectively assigned to one or more interventions (which may include placebo or other control) to evaluate the effects of those interventions on health-related biomedical or behavioural outcomes.

As referred herein, “Case Report Form” or “CRF” is a printed, optical, or electronic document designed to record all the protocol-required information to be reported to the sponsor for each patient participating in the study.

As referred herein, CRF data refers to the information recorded on CRF, which serves as the primary data collection tool. It captures essential details about study subjects, treatments, observations, and outcomes, ensuring accuracy, completeness, and consistency with the source documents.

As referred herein, “Source Data Verification” or “SDV” is the process of ensuring that the data reported for analyses accurately reflect the source data at the clinical trial site by comparing it against the CRF data (transcription errors). SDV predominantly detects random errors and focuses on ensuring data accuracy during the trial. However, 100% SDV does not guarantee error-free results.

As referred herein, “monitoring” or “clinical trial monitoring” refers to an act of overseeing the process of a clinical trial, and of ensuring that it is conducted, recorded, and reported in accordance with the protocol, Standard Operating Procedures (SOP), Good Clinical Practice (GCP) and the applicable requirements. (ICH E6 Glossary).

As referred herein, “Risk Based Monitoring (RBM)” is a clinical trial monitoring approach that focuses resources on areas posing the greatest risk to data quality and patient safety. It is an essential component of a preventive clinical trial management strategy. The primary objectives of RBM are to identify, assess, control, communicate, and review risks associated with the clinical trial throughout its life cycle.

As referred herein, “type of monitoring” refers to different approaches or methods used to oversee, track, and evaluate activities, processes, or systems. Various examples of types of monitoring are remote monitoring, on site monitoring, risk-based monitoring, centralized monitoring, safety monitoring, audits, and inspection. It can also be classified as low, medium, and high risk monitoring.

As referred herein, “level of monitoring” refers to the extent and depth of oversight, observation, and scrutiny applied to a particular activity, process, or situation. It encompasses various aspects such as frequency, intensity, and methods of monitoring to ensure that objectives are met, risks are managed, and performance is optimized.

As referred herein, “risk profile” refers to an assessment of the potential risks associated with conducting a specific clinical study. It involves identifying, evaluating, and managing various risks that may impact the trial's success, patient safety, data quality, and overall integrity.

The term “dataset” may be used broadly to refer to any data or collection of data, inclusive of but not limited to structured data (including tabular data or data encoded in JSON or other formats and so on), unstructured data (including documents, reports, summaries and so on), partial or subset data, incremental data, pooled data, simulated data, synthetic data, or any combination or derivation thereof. Certain examples are depicted or described herein in an exemplary sense without limiting the present disclosure to other forms of data or collection of data.

As used herein, “Artificial intelligence” or “AI” refers to the ability of a digital computer or computer-controlled robot to perform tasks commonly associated with intelligent beings.

As used herein, “Pre-clinical research” refers to early use of AI in pre-clinical research, impacting subsequent clinical trials.

As used herein, “Design” refers to use of AI enabling prediction of outcomes and disease progression to shape or improve Design of clinical trials.

As used herein, “Recruitment” refers to use of AI in Recruitment, which includes Enrollment, defined as the identification of eligible sites and eligible participants and onboarding them into suitable clinical trials.

As used herein, “Conduct” refers to the period following a participant's enrollment into the trial, up to the trial database lock, prior to statistical analysis.

The term “artificial intelligence unit” refers to any system that perceives its environment and takes actions that maximize its chance of achieving its goals. Artificial intelligence unit utilizes a plurality of machine learning algorithms that allow systems to automatically improve through experience and self-learning.

The term “feature” as used herein in relation to machine learning and pattern recognition, represents or refers to an individual measurable property or characteristic of a phenomenon. Features are usually numeric, but structural features such as strings and graphs are used in syntactic pattern recognition. The concept of “feature” is related to that of explanatory variables used in statistical techniques such as linear regression.

As used herein “anonymization” refers to the process of turning data into a form that does not identify and recognize individuals. Anonymization breaks the link between data and a given participant so that the participant cannot be identified, directly or indirectly (e.g., through cross-referencing), from their data.

Business problem: With the advent of hybrid and decentralized clinical trials, data is sourced from multiple discrete channels. The clinical monitor of the clinical trial must ensure that the data originating from these channels is valid, maintains its integrity, and is accurate. One of the methods used in “traditional” site-focused clinical trials is that of monitoring of the sites through which data is sourced. Therefore, with patients and the caregivers (both professional and non-professional) now sourcing data directly for a clinical trial, there is a need for an innovative solution to focus on the sources of high risk within the data collection process.

Business solution: Risk Based Monitoring (RBM) is a methodology used to help balance the level of effort, time, and cost associated with monitoring data and the sources of the data with the associated risk of either the level of monitoring required, or the type of monitoring (for example remote monitoring versus on site monitoring). The following are examples of few of the risks associated with data collected either on site or in a remote setting (possibly the subject's home or a local healthcare provider):
Is the data within established tolerances
Is the data trending over time showing a divergence from the “norm”
Is the data always submitted on time and is there an indication of trial abandonment or is the patient engaged
Has the patient submitted erroneous data in the past.
   The fundamental ways to ensure accuracy, consistency and the integrity of the data is through site visits and monitoring visits with sites where an expert from a Contract Research Organization (CRO) or pharmaceutical company conducting the clinical trial will go out to a site and look at documents, processes, procedures, and the data that has been generated from the interactions between the site and the patient. CRO is a company that provides clinical trial management services for the pharmaceutical, biotech, and medical device industries. The company may design, manage, and monitor the trial, and analyse the results. The present disclosure provides a mechanism to a formally described model, which describes the risk, and models the risk, associated with the execution of the trial at a range at every site on a clinical trial.

Technical problem: The fundamental ways to ensure accuracy, consistency and the integrity of the data in a clinical trial is through site visits and monitoring visits, that relies heavily on Source Data Verification (SDV). In risk based monitoring, as the risk parameters change over time, the type of monitoring and the level of monitoring may also change. With hundreds of clinical trials going on, the data and associated metadata related to the risk factors vary over time and there is a need to make decisions on the risk based monitoring in real-time. There is a need to plan and predict in advance, the modelling to be executed on a trial ahead of the trial starting so that the risk based monitoring may be defined well in advance.

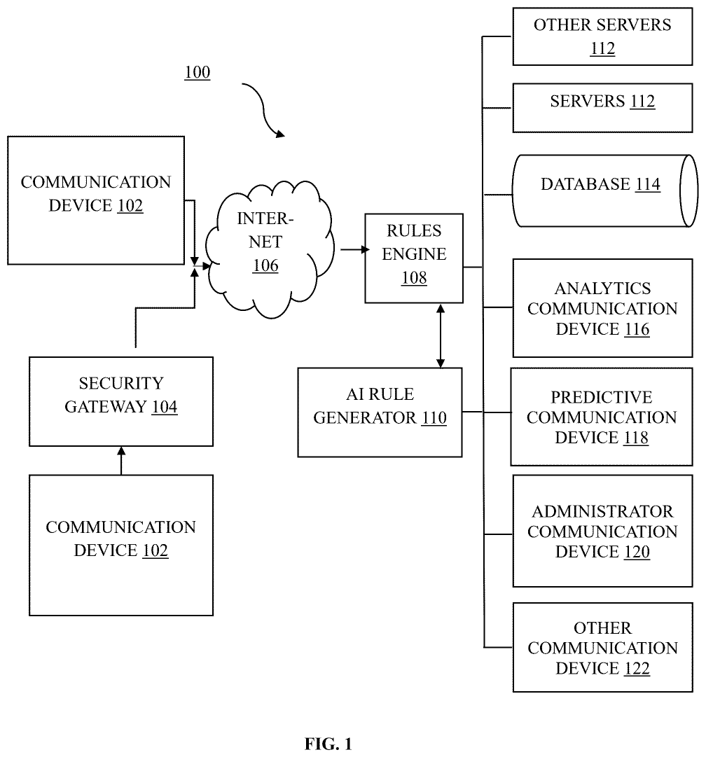

Technical solution: FIG. 1 is a diagram illustrating a clinical trial network including the main hardware components, according to one or more embodiments. Referring to FIG. 1 a clinical trial support network 100 comprises communication devices 102 linked by a security gateway 104 to the Internet 106, a rule engine 108 and an AI rule generator 110. This in turn links the communication devices 102 with servers 112, a database 114, an analytics communication device 116, a predictive communication device 118, an administrator communication device 120, and with other communication devices 122. The rule engine 108 may be configured to execute one or more rules, e.g., in a planned, scheduled, and/or ordered manner. The rules are generated by the AI rule generator 110, which uses artificial intelligence to formulate rules to enable future oriented risk based monitoring in a clinical trial. The rules enable the various workflows to be triggered. The communication devices 102 may be a patient communication device, a caregiver communication device, or a site staff communication device. Examples of the communication device include using diverse technologies for collection, processing, storage, and distribution of data such as Smart Phones, iPads, Desktop/Personal Computers, Stand-alone/On-Premise/Cloud Servers, an Electronic Medical Record (EMR), an Electronic Health Record (EHR), and the like. The data gathering may be through patients, caregivers, site staff, etc. who gather data through a diary that is maintained by the patient or the caregiver to keep a record, through an app on a phone where the patient or caregiver or the site staff enters data with regard to a patient, or through a wearable device worn by the patient which records the required vitals of the patient and sends it to the communication device. Each device and server comprises digital data processors and communication interfaces as is well known in the art.

The servers 112 are configured in hardware terms according to the specified requirements in the clinical trial network. In one example the servers have a speed in the range of 2 to 3 GHz, have in the range of 4 to 16 cores, and a memory capacity of 12 to 15 GB. However, the parameters may be different, depending on the capacity requirements.

The communication devices 102 and the servers 112 are used by the patient and by clinical trial staff at the site, and the clinical research organization. On the server side of the topology, the servers 112 include servers to model, manage, analyse, and predict the interactions between the patient and the study team (whether that study team is physical, remote, or virtual). Those devices on the server side continually interface with the database 114 of patient/trial interactions and apply algorithms to provide trends and predictions as to the likely risk based monitoring of a site in the clinical trial. The system ecosystem has various components with a suite of Artificial Intelligence algorithms developed using software such as Python and R. The database may comprise a data anonymization unit which anonymizes or pseudonymizes the inputs received by discarding the metadata, such as patient details, associated with the inputs. Anonymization may be performed to break the link between data and a given participant so that the participant cannot be identified, directly or indirectly. Such an anonymization can be performed based on rules which are preconfigured, or configured at the time of data transfer. The patient details may contribute to determining the identity or recognizing the patient. Without the patient details, it would be impossible to detect from whom (e.g., which patient, which user, etc.) the inputs are received. Key patient identifiable fields, such as, but not limited to patient name and patient ID, address, social security number, credit card information, etc., need to be anonymized before the patient medical data can be shared with research facilities. Once the data is anonymized, the patient identity is concealed such that one cannot trace or track the source (e.g., patient identity, site identity, etc.) of the medical data. In an embodiment, the data anonymization unit anonymizes the inputs by removing facial detection information and biometrics information from the inputs. For instance, when the one or more first images of the same patient received at different instances are combined, it may contribute to detect/recognize facial information or personal information of the user. In such a case, the data anonymization unit discards one or more first portions of the one or more first images. The one or more first portions are the portions that may assist in recognizing or identifying the user (e.g., patient). The database technology may be instantiated as an RDBMS or as an in-memory data grid spanning clusters of servers to allow for faster throughput and real-time processing of events as they occur during the execution of the clinical trial. The deployment of the database server may be in a private data centre on a secured public cloud infrastructure to allow for quick scale up during periods of intense activity in a clinical trial and where the volumes of data approach that of a data stream and will require additional infrastructure to support spikes in demand during these periods. Designing and developing such a complex computation system for predicting risks utilizes a software ecosystem/platform with robust computational infrastructure encompassing components as mentioned above.

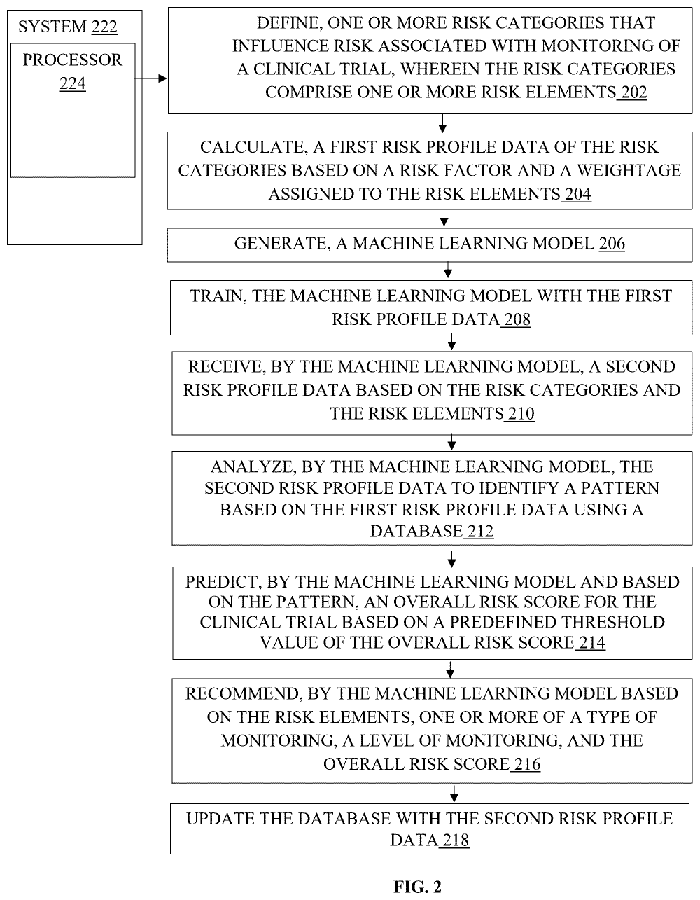

FIG. 2 shows a flowchart for a system for enabling risk based monitoring (RBM) in a clinical trial according to an embodiment. An embodiment relates to a system 222 comprising: a processor 224 storing instructions in a non-transitory memory that, when executed, cause the processor to: define, one or more risk categories that influence risk associated with monitoring of a clinical trial, wherein the risk categories comprise one or more risk elements at step 202; calculate, a first risk profile data of the risk categories based on a risk factor and a weighting assigned to the risk elements at step 204; generate, a machine learning model at step 206; train, the machine learning model with the first risk profile data at step 208; receive, by the machine learning model, a second risk profile data based on the risk categories and the risk elements at step 210; analyse, by the machine learning model, the second risk profile data to identify a pattern in the first risk profile data using a database at step 212; predict, by the machine learning model and based on the pattern, an overall risk score for the clinical trial based on a predefined threshold value of the overall risk score at step 214; recommend, by the machine learning model based on the risk elements, one or more of a type of monitoring, a level of monitoring, and the overall risk score at step 216; update the database with the second risk profile data at step 218; and wherein the machine learning model is a self-learning model comprising a feed-back layer that enables the machine learning model to learn continuously from the second risk profile data and improve the prediction of the overall risk score and monitoring decisions to enable risk based monitoring (RBM) of the clinical trial 220.

The system leverages a suite of Machine/Deep Learning algorithms for exploration of factors associated with risk and subsequently computes the scores for various types of risks. The system adopts and stacks numerous techniques for performing the tasks such as pre-processing, exploratory analysis and prediction of risk score.

Usually, the input data is obtained from multiple sources and as the data formats vary between sources depending upon how the data is stored and organized. Medical data is well known to be inaccurate, noisy, and inconsistent due to the nature of data acquisition process and diversity of the nature of data.

Therefore, the system runs its pre-processing algorithms that deal with processing both structured and unstructured data. These algorithms pre-process the structured, unstructured and image data and then forward the cleaned data to the subsequent modules for further processing and analysis. Some of the challenges in cleaning medical data and pre-processing approaches are:

### Structured Data

Data Inaccuracy—handling the incomplete, missing values can be done using traditional techniques such as imputation with mean, normal values and also with model-based approaches such as multivariate regression and k-nearest neighbor. Data Noise-reducing noise by removing erroneous data and outliers from the data by multivariate approaches using different similarity measures such as Mahalanobis and Cook.
Data Inconsistency—identified when data is input from various sources. During this time the source with the most inconsistent data can be identified and can be addressed using correlation analysis.

### Unstructured Data

Trial staff Notes/Text: For textual data the normalization can be a task for analysis of caregiver or site staff's notes and patient's laboratory reports. With normalization, the system handles some of the challenges in text processing such as:
Format/Code Conversion—data from multiple sources in various formats/codes can be collected and converted to simple format. The system incorporates Scripts for converting files in different formats to one standard format.
Eliminating Stop Words/Punctuations/Non-ASCII characters—The system incorporates regular expression scripts to eliminate the stop words, punctuations and non-ascii characters.
Identifying Stem Words—reducing each word in the text to base or root will improve the analysis of textual data. The system comprises modules for performing stemming on clinical and laboratory notes.
Lemmatization—as used herein can refer to reducing words to base form by considering the context, along with the content, and can be useful in identifying clinical, biological entities in notes or reports. Alternatively, lemmatization of words helps to tag the text.

### Medical Scans/Images

For processing medical images, the system can provide modules to perform the following tasks: Image Resize & Normalization—Images of different patients collected from different sites usually have different dimensions that are to be resized. According to various embodiments the system encompasses methods such as nearest neighbor and neural networks to perform up-scaling and down-scaling of images and also methods for transformation.
Noise Reduction—Noise in the medical images occurs due to variation in capturing and can be undesirable for image analysis. Therefore, the system comprises techniques that support reduction of various types of noises including, but not limited to, Pepper, Gaussian, and Poisson. According to various embodiments the system comprises Neural networks-based modules to suppress the noise in scanned images.
Blur—Along with noise the other major distorter for quality of an image is blur and results in affecting the accuracy of the prediction models. According to one embodiment the system comprises Kernel filters such as gaussian blur, deep neural networks, to sharpen and blur the images during the training of the prediction model. Consequently, during real-time prediction the model would have acquired resistance to blurring in the medical images.

EDA—Exploratory Data Analysis: The system also considers the synthesized results pertaining to the factors associated with data monitoring risks in clinical trials. These results can show the incidence and prevalence of the factors for risks besides providing deep insights into understanding the behaviour of risk factors for different cohorts. Such an exploratory analysis can be used by clinicians in designing the prevention and intervention strategies. Results can be rendered by rich graphical presentations through a dashboard that enables easy interpretation and assessment of risk indicators. Some of the visualizations rendered in the dashboard include, but are not limited to: Visualization of indicators such as global, local, site specific and patient specific risk categories, clinical trial risk, etc. are usually depicted using the charts speedometer, gauge meter and horizontal bar charts.

The disclosure provides a consistent computable risk score for studies, regions, sites, and patients based on a configurable system of risks and their weightings. To enable the clinical trial, the management team may determine a high-level set of input to risk, based on initial weightings from each category. As an example, the table below may be used:

TABLE 1
Risk Category Weightings

| Risk Category | Relative Risk Weighting |
|---|---|
| Global Risk | 0.2 |
| Local Risk | 0.1 |
| Site Specific Risk | 0.4 |
| Patient Specific Risk | 0.3 |

An algorithm similar to the one shown below may be enabled for each category under consideration for a clinical trial, at a study level using the parameters described above as examples. The relative risk elements and associated weightings are configured by the clinical trial management team as part of the setup of the trial.

$$categoryRiskProfile = {\sum }_{i = 1}^{i = n} riskFactor ( i ) * riskWeighting ( i )$$

Where n is the number of sub-category risks factors that the study team determines to play into the overall risk of the trial.

### Equation 1—Generic Category Risk Profile

The risk weighting may be used to generate a consistent risk measurement, and may also be used to drive and compare risk ratings and weightings across clinical trials in a consistent manner. This may then be used to refine the “starting risk point” for trials into the future.

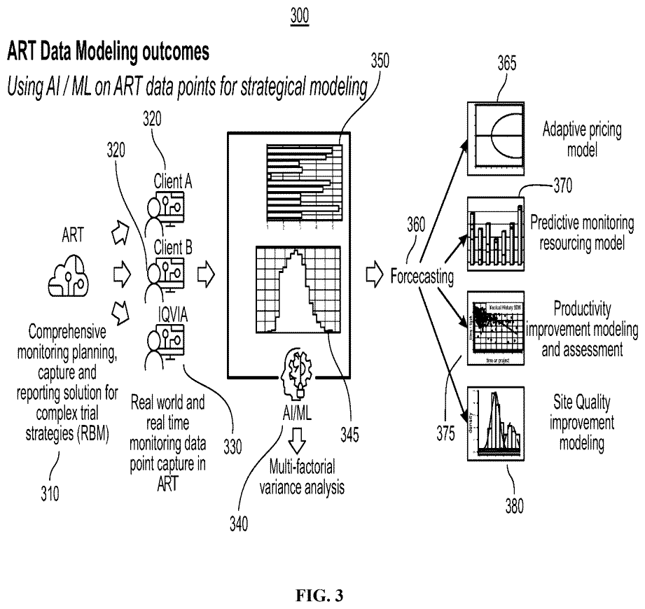

In FIG. 3, activity review tool (ART) data modeling outcomes 300 are illustrated. The ART data modeling outcomes 300 include comprehensive monitoring, planning, capture, and reporting of complex trial strategies 310. The planning can occur at the reporting visit site and can include the protocol risks, risk mitigation plan and event schedule. The site monitoring review will include identifying a to-do list to be completed on that visit. The site monitoring review will also include generating completion times data for the completed tasks and also noting the tasks completed that also include the SVR and CTMS. Further, the site monitoring review also includes providing progress reports in real-time on the completed tasks and on the workflow at the site. Further, the reporting will also include reporting any unfinished tasks at the site that need to be completed on a future visit.

Referring to FIG. 3, a real world and real-time monitoring and complex trial strategies 310 is also shown. As such, all of the tasks that need to be completed can be reported back to the reporting visit site. Further, the tasks that are completed, and the times in which the tasks are completed, including the types of tasks completed (SRV/CTMS/PD etc.) are reported in real-time. The complex trial strategies 310 will occur for a multitude of clients 320, 330 (A, B, Iqvia). Once the site monitoring plan is put together, the client/user can access the site monitoring plan on his/her mobile device, and then capture data points in real-time, and report the data points back to the reporting visit site in real-time.

In FIG. 3, a multi-factorial analysis 340 is illustrated. The multi-factorial analysis 340 is shown to include AI/ML in real-time. The AI/ML strategic models 345, 350 receive an input of the captured data points from the site monitoring review, wherein the site monitoring review was made on the risk assessment mitigation plan (RAMP). The RAMP includes mitigation actions to take in place of any encountered risks. As such, the real-time data from the completed tasks, and monitoring of the workflow, and uncompleted data is inputted into the AI/ML strategic models 345, 350. Essentially, the AI/ML strategic models 345, 350 are trained using the input captured data from the site monitoring review which the user has performed onsite.

In FIG. 3, forecasting 360 is shown as the output from the trained AI/ML strategic models 345, 350, wherein the time it takes for monitors to finish the processes they are assigned are tracked accordingly. The forecasting 360 includes adaptive pricing model 365, a predictive monitoring resourcing model 370, a productivity improvement modeling and assessment 375, and site quality improvement modeling 380. The adaptive pricing model 365 can refer to the pricing of putting together the site-monitoring plan based on the protocol risks, event schedule, and RAMP discussed above. Further, the adaptive pricing model 365 can also include the cost of executing the site monitoring onsite. In other words, the cost can refer to going through the subject data, the to-do list, and also generating the completion times, data, and completing the checklist for the tasks completed and SVR and CTMS related tasks. In addition, the adaptive pricing model can also take into account the progress reports to the reporting visit site and the cost for completing the unfinished tasks on subsequent visits.

Referring to FIG. 3, the predictive monitoring resource model 370 will refer to the planning that occurs at the reporting visit site, wherein the planning includes the protocol risks, the calendar which customers agreed upon, and the RAMP that goes into the planning phase. In addition, the unfinished tasks that occur onsite which need to be completed on subsequent visits also can be included into the predictive monitoring resource model 370.

In FIG. 3, the productivity improvement modeling and assessment 375 includes identifying the efficiency in which the tasks are completed onsite via the site monitoring review and the efficiency of the monitoring workflow. The productivity improvement modeling and assessment 375 also can identify the completion times data and the time it takes to complete the tasks on the to-do list. In addition, the productive improvement modeling and assessment 375 can identify how many of the tasks are completed in comparison to how many of the tasks are not completed and need to be completed on a subsequent visit.

With respect to FIG. 3, the site quality improvement modeling 380 can include improving the efficiency of completing the tasks on the to-do list and the efficiency of the monitoring workflow. The site quality improvement modeling 380 can also include improving the time required to provide the progress reports in real-time to the reporting visit site. The site quality improvement modeling 380 can also involve improving the monitoring of the workflow for the user while the user is onsite performing the site monitoring review.

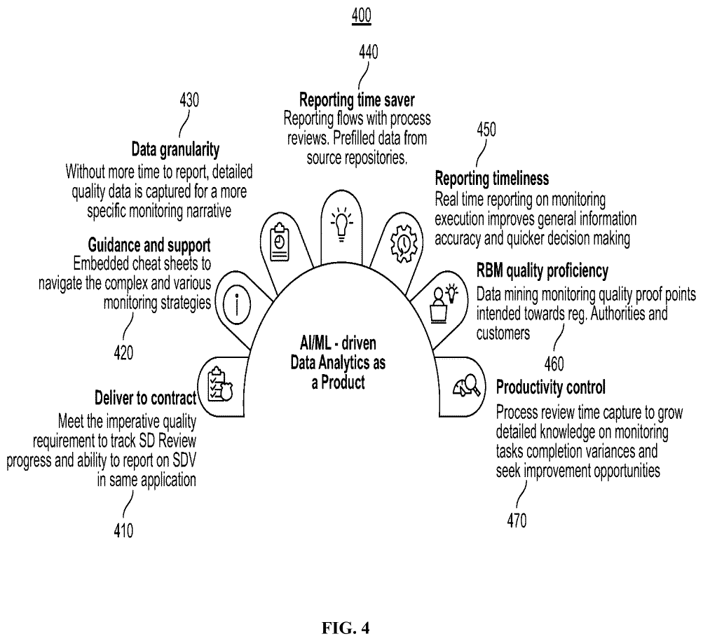

In FIG. 4, chart 400 illustrates the benefits that the system described in FIGS. 1-3 can provide. The chart 400 involves AI/ML driven data analytics as a product. Deliver to contract 410 is shown. With deliver to contract 410, the imperative quality requirement involves tracking source data review progress. The deliver to contract 410 also includes the ability to report on source data variance in the same application. The variance in the source data review is identified.

Referring to FIG. 4, guidance and support 420 is also provided. The guidance and support 420 includes embedded cheat sheets to navigate the complex and various monitoring strategies. The completed tasks and uncompleted tasks are reported in real-time. The completion times of the tasks are provided in real-time. As such, a constant reporting of the site monitoring onsite is reported to the reporting visit site in real-time.

In FIG. 4, data granularity 430 is also illustrated. With data granularity 430, without more time to report the data at the site monitoring, detailed quality data is captured in real-time for a more specific monitoring narrative. The completion times of the data and tasks are reported in real-time with the clock stop feature at the site monitoring review site. The checklist of the tasks in relation to the data are completed and reported in real-time.

Referring to FIG. 4, reporting time saver 440 is shown. The reporting time saver 440 reports flows with process reviews. The site monitoring workflow is reported in real-time to the reporting visit site. Tasks that are being completed from the to-do list are reported, and tasks that are not completed are reported in real-time. Prefilled data from source repositories are also included. The prefilled data from source repositories can include the completed data onsite from source repositories that are onsite at the site monitoring review.

In FIG. 4, reporting timeliness 450 is illustrated. Reporting timeliness 450 includes real-time reporting on monitoring execution. This improves the accuracy of the information reported and results in quicker monitoring based decision making. The completion times data of the completed tasks is accurately reported in real-time. The tasks that are not completed are reported on task reports and reported to the reporting visit site in real-time. The progress and efficiency of the monitoring of the workflow is more accurately reported in real-time.

With respect to FIG. 4, risk based monitoring (RBM) quality proficiency 460 is also shown. The RBM quality proficiency 460 includes data mining monitoring quality. The RBM quality proficiency 460 also includes proof points intended toward reg. (regulating) authorities and customers. Overall, the RBM quality proficiency 460 overall will include the mined data at the site monitoring site and reports of the mined data to customers and authorities.

Referring again to FIG. 4, productivity control 470 is also illustrated. The productivity control 470 will include process review time at the site monitoring site. The review time it takes to capture and grow detailed knowledge on the data, and completed tasks onsite, is included in the productivity control 470. The productivity control 470 also includes monitoring task completion variances. The task completion variance includes the tasks that are completed on the to-do list and the tasks on the to-do list that could not be completed and need to be completed on subsequent visits. The improvement opportunities within the productivity control 470 include identifying methods and systems to monitor the workflow and complete the tasks on the to-do list more efficiently. The productivity control 470 can also include the efficiency in which the completed tasks and completion times data are reported in real-time to the reporting visit site.

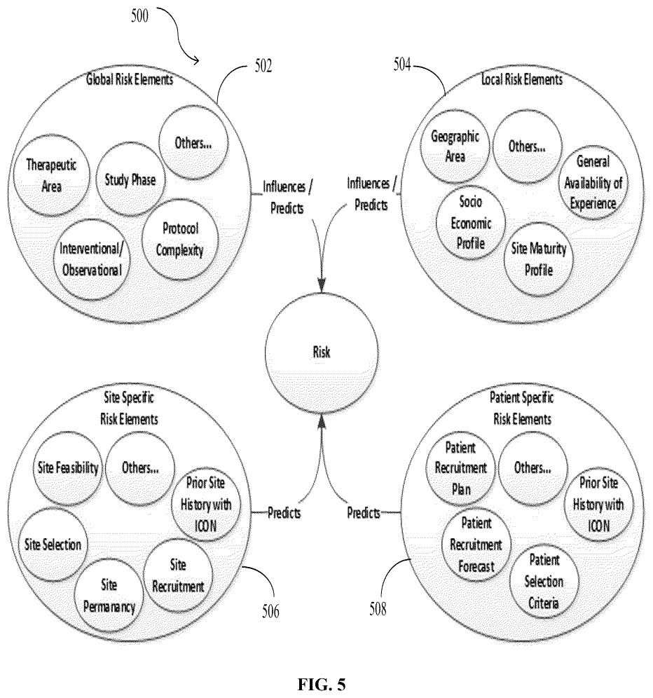

Technical details specific to the technical solution: In FIG. 5 diagram 500 illustrates some of the categories of risk profiling along with some examples of the risk elements that may apply to each category. As a clinical trial starts, some of its attributes can be used to generate a “starting risk profile”. Examples of some of the factors/attributes that would influence the risk include:
a. Global Risk Elements 502
   i. Therapeutic Area
      1. Oncology, Heart, Respiratory, Vaccine, Women's Health, Rare, Orphan
   ii. Study Phase
      1. Phase I, II, III, IV
   iii. Protocol Complexity
      1. The more primary and secondary outcomes the more complex the trial becomes and the harder it is for the sites to execute all of the additional steps required for the various outcomes defined in the protocol.
   iv. Interventional/Observational
      1. Interventional are higher risk to begin with as patient safety and efficacy are primary constraints. In an observational study, patients are not given therapy as part of the trial.
b. Local Risk Elements 504
   i. Geographic Area
      1. Some geographic areas are at higher risk, perhaps due to civil/military unrest, political chum, the geopolitical situation around the area which may indirectly make it difficult to execute the trial, and potentially limit access to sites. The infrastructure at various areas will also be heterogeneous which may also influence the risk/methodology chosen for RBM.
   ii. Socio Economic Profile
      1. Example data elements here include relative ranking on area wealth, education, crime, employment rates, transport infrastructure, communications infrastructure, health services.
   iii. Site Maturity Profile
      1. Elements that may affect site maturity: number of studies completed, studies in a specific therapeutic area, patient throughput, years open, site relationships to other health campuses locally, staff chum, staff experience, etc.
   iv. Site Experience Profile
      1. Similar in some ways to site maturity but would reflect the level of expertise in the country/area, in the specific sites under consideration and more widely. The expertise would be the experience of clinical trial staff and caregivers available in the field for a particular clinical trial.
c. Site Specific Risk Elements 506
   i. Site Feasibility
      1. It includes how able is the site and how far ahead it is of the feasibility threshold. Example elements include equipment, ease of patient access, investigator experience, staff availability and experience, key opinion leadership, etc.
   ii. Prior History
      1. Number of studies performed, relative ranking in terms of execution to plan (patient recruitment, deviations, error rates, late/incomplete data submission, response times).
   iii. Site Permanency
      1. Is the site a popup site versus one in place and aligned to a health facility for an extended period of time and also involvement with local communities of care.
   iv. Site Selection
      1. A measure of the contract negotiation process and how onerous/difficult it was with the site. Sometimes an indicator of the future relationship.
   v. Site Recruitment Plan
      1. Ranking of the site relative to other sites in the study.
d. Patient Specific Risk Elements 508
   i. Patient Recruitment Plan
      1. Is the patient recruitment plan more/less aggressive than prior studies—again matched across therapeutic area, inclusion/exclusion criteria, protocol complexity, etc.
   ii. Prior History
      1. Involvement of the patient in other trials and experience of what it takes to engage to the end of the trial.
   iii. Patient Selection Criteria
      1. How complex is the selection process and, based on the criteria (age, location, etc.), how likely is the patient to engage given that they are selected.
   iv. Patient Recruitment Forecast
      1. What is the forecast compared to the feasibility assessment?How can the site support the forecast in terms of staff, facilities, equipment, access, etc.

Clinical trials run in various phases and high risk phases might be phase three when it's being tested on a larger group of people, also there is phase one and two as well for a particular therapeutic area which could be high risk because maybe a trial doesn't get beyond phase one or two and phase one or two is where safety, efficacy, and dosage are looked at prior to going to a mass market clinical trial, which is usually phase three. Further, in an embodiment, there may be local risk elements to a geographic area, economic profile of the patients attending the sites in those areas, the general availability of experience in those geographic locations and so on. The site specific risk elements may be that the site did not rank highly in terms of its feasibility, execution and hosting of the clinical trial and there may be a history prior to this trial with that site and how the site selection process had gone. Further, there may be factors such as contract negotiation and recruitment for a particular site. With regard to patient specific elements, the prior history with a particular patient may be considered regarding the inclusion and exclusion criteria in a protocol. There may be various influences and predictive elements that go into the model to provide a mechanism to model the risk using a semantic model, and with more data and trials, AI and machine learning build the models into the future.

The table below provides an example of risk classification, according to one or more embodiments.

| Risk elements | High/critical Risk | Medium Risk | Low Risk |
|---|---|---|---|
| Site | Subject recruitment | Subject screening failure | Electronic data |
| performance | randomization rate | rate | change capture |
| risks/KRIs | protocol deviation/violation | Data Clarification Form | history (site level |
| | per subject | (DCF) generation rate & | per X efficiency |
| | CRF completion/violation | open queries status (Site | data points) |
| | per subject | level) A DCF or data | |
| | subject discontinuation -site | query form is a | |
| | wise percentage discontinuation | questionnaire | |
| | rate subject visits- | specifically used in | |
| | outside window. | clinical research. | |
| Data quality | Missing critical data-patient | Data reporting-Digit | Missing data |
| risks/CSM | eligibility, critical efficacy, | preference (data rounding) | points-key |
| | safety data points- e.g. BP, | Data related to medical | demographics |
| | HbA1c; | history and concomitant | |
| | key efficacy and point data- | medications | |
| | data outliers or data | | |
| | inconsistency-e.g., seizure | | |
| | frequency (epilepsy study), | | |
| | HbA1c (diabetes study); | | |
| | Correlation check site level | | |
| | for critical variables Fasting | | |
| | Blood Glucose (FBG) and HbA1c; | | |
| | AE/SAE under or over | | |
| | reporting (site level). | | |
| Patient | Review of listing of AE/SAE | | |
| profile/ | for patients, review of | | |
| Listing | listing of critical efficacy | | |
| | data points for patients | | |

A combination of the factors above, with Site and Patient specific factors, would apply an overall “risk” to a clinical study and would determine the default approach to be taken by the study staff tasked with ensuring compliance—for example the Clinical Research Associates (CRAs) or Clinical Trial Assistants (CTAs). A CRA also called a clinical monitor or trial monitor, is a healthcare professional who performs many activities related to medical research, particularly clinical trials, and a CTA is responsible for supporting clinical drug research and development tasks. In an embodiment, within the Predictive Communication Device 118 and the Administrator Communication Device 120 the user may create a model of the risk and the influences that certain metadata has on the management of risk based monitoring. Examples of the method of monitoring based on the risk may be as follows:
On Site monitoring where up to 100% Source Data Verification (SDV) may be carried out
Remote Monitoring where up to 100% SDV may be carried out
No Monitoring
And variations on any of the above
   In a particular example, at the start of a trial, based on the factors above, the monitoring staff may default to 100% SDV on on-site monitoring as it may be decided to visit a site because the risk associated with the specific site and the therapeutic area and the geographic location for the particular disease for the trial is high. Over time, and depending on the performance of the sites/patients in submitting accurate data in a timely manner, monitoring may move to lower percentages of on-site SDV, such as 50% on-site SDV, and thereafter through to remote monitoring. There may be sites that indicate a trend or may indicate that there is a growing risk or may indicate that there is a specific issue that needs to be looked at, but the particular nature of the indication may be perhaps one that can be monitored remotely and there may not be a need to physically send somebody to that site to do the monitoring associated with the data that is being gathered. Further, based on the performance of the data gathering process the study team may determine that no monitoring is required for certain sites/patients because the data that is coming into the electronic systems is correct, accurate, complete, contemporaneous, and as required for the clinical trial.

In another embodiment, based on the modelling performed in the Predictive Communication Device 118 and the Administrator Communication Device 120 the following example table of risks and weighting may result for the Global Risk. A similar table may be created for Local, Site, Patient and other risk categories that may arise as part of the trial design or be identified as the clinical trial proceeds.

TABLE 1
Global Risk Profile Risks and Weightings

| Risk Element Description | Risk Factor | Relative Risk Weighting |
|---|---|---|
| Therapeutic Area: Oncology | 0.9 | 0.6 |
| Study Phase: III | 0.4 | 0.3 |
| Interventional Study | 0.7 | 0.05 |
| Protocol Complexity: Low | 0.3 | 0.05 |

An instance of the general algorithm shown above may be instantiated for each category, with the Global Risk Profile used to drive the example below. The relative risk elements and associated weightings may be configured by the clinical trial management team as part of the setup of the trial as shown in Table 2—Global Risk Profile Risks and Weightings.

$$globalTrialRiskProfile = {\sum }_{i = 1}^{i = n} riskFactor ( i ) * riskWeighting ( i )$$

Where n is the number of trial-level risk factors that the study team determines, play into the global elements of risk associated with the trial.

### Equation 2—Global Trial Risk Profile

From the algorithm and the data input from the table of risk factors and weightings, the globalTrialRiskProfile is calculated to be 0.71. At this point the clinical trial team may make a determination that any trial with a globalTrialRiskProfile value >0.6 begins with 100% on-site SDV.

As mentioned above, a similar geographic or local set of factors may be used to further refine the starting point for local variations.

$$localTrialRiskProfile = {\sum }_{i = 1}^{i = n} riskFactor ( i ) * riskWeighting ( i )$$

### Equation 3—Local Trial Risk Profile

Those geographic or local factors may influence the global value to further determine local activities with the following examples in Table 3—Local Risk Profile Risks and Weightings:

TABLE 2
Local Risk Profile Risks and Weightings

| | | Relative Risk |
|---|---|---|
| Risk Element Description | Risk Factor | Weighting |
| Geographic Area: US | 0.05 | 0.1 |
| Patient Socio Economic: Elderly | 0.9 | 0.2 |
| Established Sites | 0.05 | 0.1 |
| Site Personnel Expertise/Experience | 0.5 | 0.6 |

From the algorithm the localTrialRiskProfile calculates to 0.49.
Repeating this across the various categories results in a series of risk values associated with risk categories.
In an example, the clinical trial managers may then determine the overall risk for a trial and use that to determine monitoring decisions relative to the associated risk. The following table may be used as input to the equation:

$$clinialTrialRiskProfile = {\sum }_{i = 1}^{i = n} categoryRiskFactor ( i ) * riskWeighting ( i )$$

Where n is the number of risk categories that the study team determines, play into the clinical trial risk profileR

### Equation 1—Clinical Trial Risk Profile

To generate a risk score for the clinical trial, which may be used in support of determining some threshold values in terms of RBM, the table below may be used:

TABLE 3
Clinical Trial Category Risks and Weightings

| Risk Category | Category Risk | Relative Risk Weighting |
|---|---|---|
| Global Risk | 0.71 | 0.2 |
| Local Risk | 0.49 | 0.1 |
| Site Specific Risk | 0.32 | 0.4 |
| Patient Specific Risk | 0.6 | 0.3 |

Feeding these values into Equation 4—Clinical Trial Risk Profile results in an overall Clinical Trial Risk of 0.499. The leadership team may determine for the default action to only carry out 100% SDV when the combination risk profile exceeds 0.5. The leadership may further determine that the default action for any location, site, or patient is to now go to 50% remote monitoring.

Technical result: Accordingto an example, to demonstratethe combinatorial explosion that results in the combination of risk categories on their own (without the added combination of the risk elements), Table 5—Example Combinations for Clinical Trial Risk shows the case for 4 categories, 3 geographic locations, 6 sites, and 12 patients.

TABLE 4
Example Combinations for Clinical Trial Risk

| | | US | | | | EU | | | | Asia | | | |
|---|---|---|---|---|---|---|---|---|---|---|---|---|---|
| | | Mature Site A | | Immature Site B | | Mature Site C | | Immature Site D | | Mature Site E | | Immature Site F | |
| | | | Not | | Not | | Not | | Not | | Not | | Not |
| | | Risky | Risky | Risky | Risky | Risky | Risky | Risky | Risky | Risky | Risky | Risky | Risky |
| | Risk | Patient | Patient | Patient | Patient | Patient | Patient | Patient | Patient | Patient | Patient | Patient | Patient |
| | Weight | 1 | 2 | 3 | 4 | 5 | 6 | 7 | 8 | 9 | 10 | 11 | 12 |
| Clinical | | 0.551 | 0.401 | 0.656 | 0.506 | 0.536 | 0.371 | 0.731 | 0.536 | 0.589 | 0.394 | 0.685 | 0.568 |
| Trial | | | | | | | | | | | | | |
| Global | 0.2 | 0.71 | 0.71 | 0.71 | 0.71 | 0.71 | 0.71 | 0.71 | 0.71 | 0.71 | 0.71 | 0.71 | 0.71 |
| Local | 0.1 | 0.49 | 0.49 | 0.49 | 0.49 | 0.64 | 0.64 | 0.64 | 0.64 | 0.72 | 0.72 | 0.72 | 0.72 |
| Specific | 0.4 | 0.3 | 0.3 | 0.6 | 0.6 | 0.3 | 0.3 | 0.6 | 0.6 | 0.3 | 0.3 | 0.6 | 0.6 |
| Sites | | | | | | | | | | | | | |
| Specific | 0.3 | 0.8 | 0.3 | 0.75 | 0.25 | 0.7 | 0.15 | 0.95 | 0.3 | 0.85 | 0.2 | 0.77 | 0.38 |
| Patients | | | | | | | | | | | | | |
| 100% SDV | | 0.5 | 0.5 | 0.5 | 0.5 | 0.5 | 0.5 | 0.5 | 0.5 | 0.5 | 0.5 | 0.5 | 0.5 |
| OnSite | | | | | | | | | | | | | |
| 50% SDV | | 0.4 | 0.4 | 0.4 | 0.4 | 0.4 | 0.4 | 0.4 | 0.4 | 0.4 | 0.4 | 0.4 | 0.4 |
| Remote | | | | | | | | | | | | | |

The patient data used in the example combinations for clinical trial risk as shown in Table 5 above may be anonymized. A header anonymization function can be performed on each of the set of patient identifiers to generate a corresponding set of anonymized fields. An anonymized medical scan can be generated by replacing the subset of fields of the header of the medical scan with the corresponding set of anonymized fields. A text anonymization function can be performed on the subset of patient identifiers to generate corresponding anonymized placeholder text for each of the subset of patient identifiers. An anonymized medical report can be generated by replacing each of the subset of patient identifiers with the corresponding anonymized placeholder text. The set of anonymization functions can include at least one hash function, for example utilized to hash a unique patient ID such as ‘Risky Patient 1’ or ‘Not Risky Patient 2’ as used in Example 5 above. The anonymization can be done in a number of ways including, but not limited to, blanking or masking out characters in the DICOM (Digital Imaging and Communications in Medicine) header, replacing characters in the DICOM header with non-identifying characters, substitution, encryption, transformation, etc. Depending on the anonymization methods, de-anonymization, or partial de-anonymization, may be possible. For example in a clinical trial, if a patient is experiencing unacceptable side effects, it would be desirable to de-anonymize their clinical trial data to determine whether the patient was taking a placebo or a drug. The access control would be necessary so that only those users with certain privileges would be allowed to de-anonymize the data.

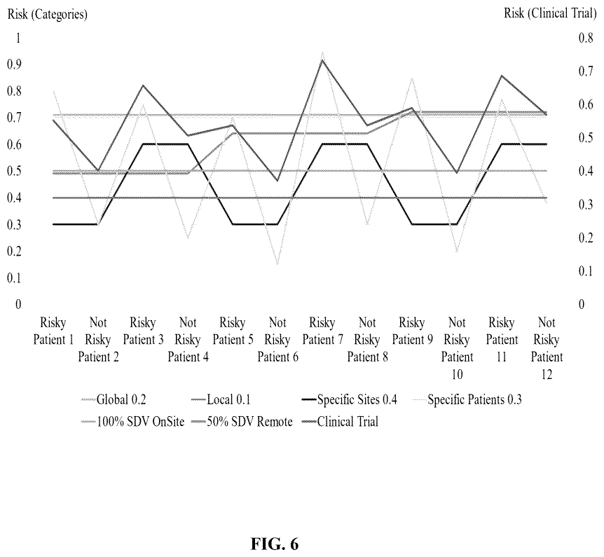

FIG. 6 is a plot generated by a prediction model employed by the Predictive Communication Device 118 and the Administrator Communication Device 120 of the clinical trial network 100, indicating a suggested starting risk profile across global locations based on global and local risk attributes according to one or more embodiments. In an example, as a default and defined by the clinical trial management team, 100% SDV Onsite may be considered once the clinical trial risk rating (Clinical Trial risk line in the graph) exceeds 0.5. When the risk rating is 0.5 or lower, 50% SDV Remote may be enabled.

In the example above:
Due to the combined global and local risk factors, Asia may start with 100% onsite SDV.
The EU which is between the two thresholds at 0.4544 may start at 100% onsite SDV, but the study design team may decide to go for 100% SDV Remote (more decision thresholds may be created by the leadership team depending on the need and to give more granular decision points).
The US may begin at 50% SDV Remote as the risk rating is 0.3497 and below the combined threshold of 0.5.
   From the data the clinical trial managers may also decide how to refine the monitoring decision to individual sites or even patients, based on the thresholds and the blended risk.

The listing of risk elements in the various clinical trial risk profiles, global, and local profiles are exemplar and simplified for illustration. The number and type of risk parameters, and associated Risk Factors and Weightings may be more extensive, possibly numbering in the hundreds or thousands given the number of sites, investigators, and patients involved in a clinical trial and finely tuned to the needs of a specific clinical trial. A different weighting may be assigned to the risk categories based on a pattern of outcomes of historical clinical trials by the Predictive Communication Device 118 and the Administrator Communication Device 120 and the weighting may be assigned to factor values before generating the site risk score. Further, a different weighting may be assigned to the risk categories at different time periods within the clinical trial, by the Predictive Communication Device 118 and the Administrator Communication Device 120, based on the changes to the risk category and the risk elements as the trial progresses. A different weighting may be assigned to the risk elements, by the Predictive Communication Device 118 and the Administrator Communication Device 120, based on another pattern of outcomes of historical clinical trials. Further, a different weighting may be applied to the risk elements at different time periods within the clinical trial, by the Predictive Communication Device 118 and the Administrator Communication Device 120 of the clinical trial network 100. There may be an additional set of risk elements to be defined for a specific site to determine whether and what kind of monitoring is needed to ensure a risk managed compliance approach.

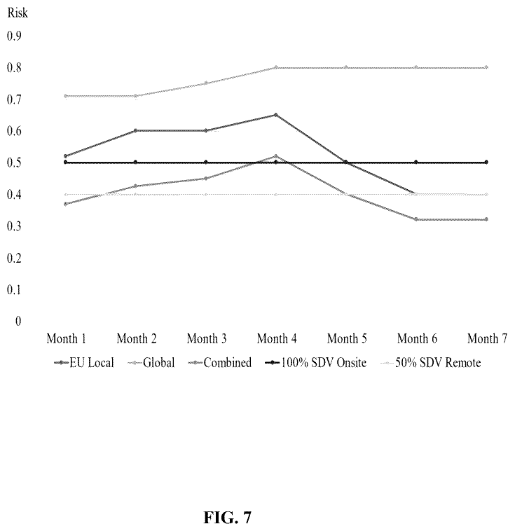

FIG. 7 is a plot showing temporal risk according to one or more embodiments. As the clinical trial progresses, events may arise that change the risk rating at a:
Global level (perhaps due to a rise/reduction in the number of Serious Adverse Events (SAE) in the trial)
Local level (if for example, there is a local weather/environmental event causing disruption)
Site/Patient level (if for example, a site is proving to be a rising risk due to timeliness or quality of data being received, or if individual patients, or patients at a specific site, are showing a higher risk of abandoning the trial, or if the site is temporary or multi-functional in nature, where a mobile van might park at a supermarket to provide a basic clinical trial site, or where the site might also be a pharmacy or other local care centre).
   The cases may force a change in the monitoring approach at any level as illustrated in FIG. 7. In this case it is observed that:
The Global risk raised to 0.8 from 0.71 due to an influx of SAEs across the study.
The EU Local risk varied from month to month due to site/patient issues arising and being dealt with in turn.
   During Month 2, the combined risk went over 0.5 signaling that a change in approach may be needed to move from 50% SDV Remote to more monitoring. This continued to rise through Month 4 where the sites warranted 100% onsite SDV due to the combination of SAEs at a global level and some local issues. Once local changes were made, they resulted in lowered risk and a return to 50% SDV Remote.

The above mentioned examples describe how decisions on risk based monitoring may be made in real-time using models, data, and algorithms. There is a need to plan and predict the modelling by the Rules Engine 108 and AI Rule Generator 110, to be executed on a trial ahead of events and proactively before the models, data and algorithms indicate a change taking place. This may be carried out by the following methods:
The plot in FIG. 7 may be used to determine the trends and acceleration of change to predict upcoming actions by the Predictive Communication Device 118 that need to be taken to proactively by the Analytics Communication Device 116 and the Administrator Communication Device 120 to prevent a risk situation developing (such as increasing SDV or on-site monitoring).
Using as input to the models the concept of plans for site and patient recruitment and their associated metadata to drive “future-oriented” data and visualizations to aid in planning.

Technological advancement: In the example data sets used in Table 2, Table 3, Table 4, and Table 5, the data used may reflect historical data—data that originated from events that were carried out or observed sometime in the past. To get a predictive model which is independent from the event, either historic or current, the model extends to include planning data sets, such as site and patient recruitment plans and forecasts. By adding planning and forecasting risk elements to the site and patient specific categories, the form of the model takes on a temporal predictive nature. Planning and forecasting carried out by the Predictive Communication Device 118 and the Administrator Communication Device 120, gives estimates of when certain sites and types of patients may be recruited into the study, which will help predict risk ahead of the trial starting. With the example model above, knowing what and when sites are feasible, selected and then recruited may aid in defining the risk based monitoring to be done potentially months in advance, with minor corrections to be made as data feedback is fed back into the model as the clinical trial proceeds.

In an example, for a particular therapeutic area in a particular location, the risk elements that prove to be influential and predictive in nature to the use of a site in the clinical trial, over time, with AI carried out by the Predictive Communication Device 118 and the Administrator Communication Device 120 get better in terms of predicting the model. The risk may change over time and the trends may change in terms of increasing issues to impinge on thresholds and the overall risk. The system of the clinical trial network 100 adapts as the clinical trial progresses and as new data emerges, AI machine learning as employed by the Predictive Communication Device 118 may ease the burden in terms of defining the models into the future and using that as a mechanism to assist in selecting sites, selecting patients, selecting therapeutic area, selecting regions, and identifying sites that are high risk or low risk and evolve to determine by the Administrator Communication Device 120, the type and level of monitoring required for a particular site based on the data. The result of including these planning/forecasting elements to the model is that the graph in FIG. 7 becomes a forward looking graph (how the trial is likely to proceed based on plans/forecasts) rather than a graph that is historic (showing how the trial has proceeded based on events that have already taken place).

As an example, the model may be based on a network of interconnected nodes representing the risks. The edges represent the influence one node (risk) has on another node (risk). This may be represented in Resource Description Framework (RDF) and stored in a graph or relational database. The node used in the subject of an RDF statement may be a blank node. The resources indicated by blank nodes are anonymous resources. The blank node or bnode may represent a subject about which other than the relationship nothing else is known. The ontology that may be built would be customizable for a study—initially may be by a Contract Research Organization (CRO), but stored and referenced in such a way that the model may be reused and enriched by subsequent application in more and more studies. The data that would act as the training data set for the initial implementation may use the results of previous studies. An exercise may be undertaken to gather the metadata associated with the four risk categories listed above (global, local, site specific, patient specific) and applied to the outputs of site monitoring as performed (usually 100% on site, 100% source data verification—SDV). Some of the data to be used here includes: the level of findings garnered from 100% SDV on 100% site visiting, the relative ranking of those sites received during feasibility assessment, etc. That outcome may define the causality between deviations and poor output from a site relative to the therapeutic area, region, site, and patient variables mentioned above. Once the metadata is gathered, an artificial intelligence (AI)/machine learning (ML) model may be trained with the metadata using the Analytics Communication Device 116 and the Administrator Communication Device 120 of the clinical trial network 100. The AI/ML model synthesizes large data volumes. In addition, the AI/ML predicts output in a substantially reduced time period, wherein the time period can be just seconds. The AI/ML model predicts the output in a substantially reduced timeframe than what could be performed by humans in a cloud-computing platform.

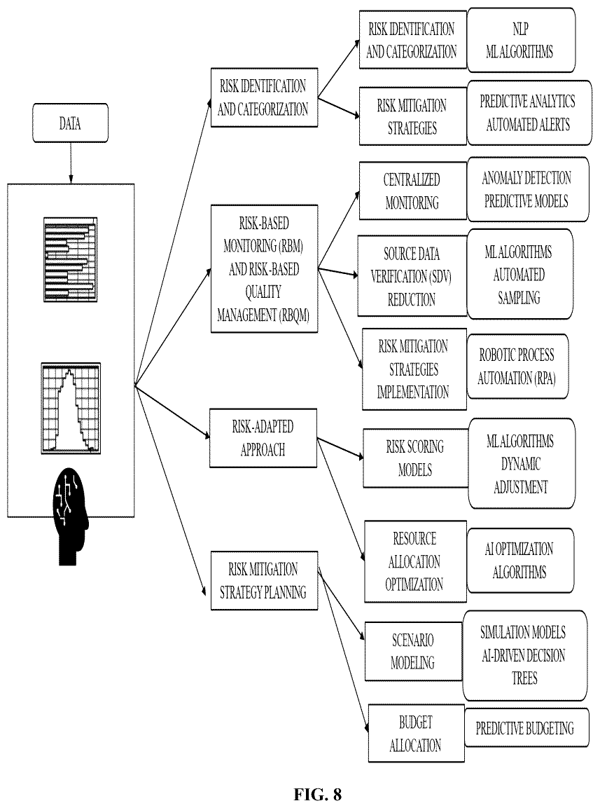

AI-driven decision Artificial Intelligence (AI) plays a pivotal role in enhancing risk assessment processes within clinical trials at various stages. FIG. 8 shows use of the AI unit of the system for risk assessment, according to one or more embodiments. The first stage involves the identification and categorization of risks, where AI applications like Natural Language Processing (NLP) techniques analyse unstructured data, such as medical literature and adverse event reports, to extract meaningful insights related to drug interactions, adverse effects, or patient outcomes. Machine Learning (ML) algorithms, including supervised learning classification algorithms and unsupervised learning clustering algorithms, aid in categorizing risks based on historical patterns and existing labelled data. This analysis helps in identifying potential risks by extracting meaningful insights from the data.

Once risks are identified and categorized, AI contributes to the implementation of risk mitigation strategies. Predictive analytics techniques are employed to forecast potential risks based on historical data and patterns, enabling proactive risk management measures. Automated alerts generated by AI algorithms serve as an early warning system by triggering alerts when specific risk thresholds are breached, ensuring timely risk response actions. Predictive analytics techniques, such as time series forecasting models like Autoregressive integrated moving average (ARIMA) or LSTM, can predict future risks based on historical data trends. Ensemble methods like Random Forests or Gradient Boosting aid in predicting risk probabilities and prioritizing mitigation efforts. Automated alerts, including threshold-based alerts and Bayesian Networks, monitor risk indicators and trigger alerts when predefined thresholds are breached, ensuring timely risk response actions.

In the context of Risk-Based Monitoring (RBM) and Risk-Based Quality Management (RBQM), AI-driven anomaly detection techniques such as machine learning algorithms like Isolation Forests or One-Class SVM detect unusual data patterns that may indicate risks. As an example, the classification of the patient's state at any instant is whether pain is being experienced at or above the specified level, and the data used to make the classification (prediction) consist of the remaining acquired data as a function of time. SVM is trained to predict the presence of the specified level of pain (or greater) from the other coincident data, and/or it is trained to predict from the other data whether the level of pain will be experienced some specified number of seconds into the future. By extrapolating the values of the other data into the future, it is also possible to use those extrapolated data to predict the future value of the patient's pain, using an SVM that is trained with concurrent other data, but by training the SVM with other data that had been evaluated at times prior to acquisition of the pain data, the extrapolation step is avoided. Because the SVM analysis is repeated for all possible pain levels, the analysis collectively endeavors to predict the current or future numerical value of pain. As SVM is trained to predict the future value of pain it may provide better trust parameters to the patient. SVM may further utilize clustering techniques, where the machine learning algorithm is provided with unlabelled or unclassified data, which leaves the algorithm to identify hidden structure amongst the cases. SVM can be used by a user interface to perform SVM training, classification and prediction, and to automatically capture and identify clusters and generate output in a user interface. SVM Classifier is a cross-platform user interface that may be used. The SVM clustering can be further combined with k-means clustering to accelerate the training and prediction of the model. One benefit of training and testing an SVM is that it will provide the caregiver with a better sense of which variables are responsible for the patient's pain and which stimulation modalities and parameter ranges would most likely effect a reduction in the patient's pain. Thus, analysis of the data concerning the patient's pain, along with the other data that may predict that pain, provides diagnostic information to the caregiver. With that information, the caregiver would reduce the time and effort needed for trial-and-error experimentation with stimulation modalities and parameter values. The caregiver may also use the diagnostic data that has been collected for a population of patients, in order to determine how subsets of patients having similar diagnostic data respond to different treatment modalities. The SVM model may predict or detect the overall risk score for the clinical trial site based on a predefined threshold value of the overall risk score. Prioritization of critical data points for verification during RBM is facilitated by AI-driven ML models, reducing the workload associated with Source Data Verification (SDV). Reinforcement learning algorithms also contribute to efficient resource allocation based on risk levels. Centralized monitoring is facilitated through AI-driven anomaly detection, which identifies unusual data patterns or outliers that may indicate potential risks. Predictive models developed using AI techniques help forecast deviations from expected data trends, enabling proactive risk management strategies.

A risk-adapted approach is facilitated through dynamic risk scoring models, including recurrent neural networks (RNNs) that continuously update risk scores based on real-time data. Online learning algorithms ensure responsiveness by learning incrementally from new data. Resource allocation optimization techniques such as genetic algorithms maximize risk management effectiveness by allocating budget and personnel, based on risk profiles.

In the planning phase of risk mitigation strategies, AI facilitates scenario modeling by generating plausible risk scenarios through simulation models. Scenario modeling and decision trees are integral parts of risk mitigation strategy planning. Monte Carlo simulation models generate plausible risk scenarios, aiding in planning and decision-making processes. Decision trees, driven by AI algorithms, recommend optimal risk mitigation actions based on scenario analysis, enhancing decision-making processes within clinical trials.

Artificial Intelligence for Prediction: In various embodiments of the claimed invention, the system as disclosed herein strives to predict the type and level of monitoring required for a site with regard to the overall risk of the clinical trial. For predicting each risk, the system employs a suite of Artificial intelligence techniques to determine a risk score with values between 0 and 1. These techniques can be trained on millions of medical records having medical, clinical characteristics of the clinical trial to attain the ability to generalize the overall risk for the trial. Pre-processed data can be forwarded to the AI Suite for learning how to generalize and predict. The Suite can have a set of machine learning techniques that learn how to preprocess the data, extract information from the texts and images and subsequently assemble the knowledge to perform risk prediction.

Representation Learning: Performance of the prediction model depends upon the quality of data pooled for training the model. Deep neural network models are trained to learn data representation for the data considered as the input. To improve the performance of the prediction model, vector representation can be adopted to denote the content in the clinical trial. Furthermore, information extracted from the various sources of data are also combined with the other characteristics of the data and are represented as vectors.

Learning & Extracting from Text Data: The AI suite of the system has neural network models of type recurrent neural networks to perform the task of extracting information from the unstructured data such as patient diary, lab reports, caregiver, or site staff's notes etc. Models can be trained to identify the clinical concepts in text and map them to the standard clinical approaches. Thereby trained models enable transformation of unstructured text into information represented in vectors.

Learning & Extracting from Image Data: The AI suite of the system comprises deep neural network models of type convolutional neural networks to perform the extraction of information from different types of scans such as ultrasound, MRI etc. Networks are trained to learn object segmentation from the scanned images. Once trained, the model has the ability to detect objects from the knowledge it has gained about image features. Upon extraction of the object from the scanned image, information about the properties of the object are represented in vectors.

### Learning to Predict the Overall Risk Score

Information extracted from the above deep neural networks can then be passed to the stacked neural networks with deep hidden layers. These layers have a large number of nodes with non-linear activation functions and thus have the ability to capture the non-linear association with the various data characteristics of the risk elements. Projection of the risk element characteristics to the higher dimension will enhance the opportunity to better understand the association between different characteristics. Training of the model is done in the context of supervised learning. Consequently, the model's ability to identify and extract patterns from the risk element characteristics pertaining to a risk can be reliable with statistical significance. Further, training of the entire stacked deep network can be repeated to identify optimal values of epochs and batch size.

In an embodiment, the AI model used for the prediction of the risk score for the clinical trial may be an Explainable artificial intelligence (XAI) model or Explainable AI model. Explainable AI may be used to describe an AI model that provides reasoning or justification for the decisions taken by the model thus allowing for transparency in the model. xAI algorithms are programmed to describe its purpose, rationale and decision-making process in a way that can be understood by the average person. Explainable AI may address certain issues pertaining to AI-based analysis such as bias, transparency, safety, and causality. Bias refers to potentially flawed AI resulting from biased training data. xAI is often discussed in relation to deep learning and plays an important role in the FAT ML model (fairness, accountability and transparency in machine learning). As the model provides insights into taking its decisions and making predictions, determination of overall risk of the clinical trial by this model is better understood by the users of the model and the management team. Explainable AI model may also provide better trust parameters for the patients and the management team, as the model provides more transparency to the inner working of the model, which makes it better understandable to humans. Explainable AI technique provides an effective manner of illustrating the back-end reasoning process of the AI system at a considerably granular level, which allows the user to make an informed decision or accept a decision made on behalf thereof. An ontology may be created and associated with the clinical trial data. Historical clinical trial data may be parsed frequently and curated to form ontologies and link the data with the ontologies. Unsupervised learning, for example, k-means clustering, can be used to cluster feature vectors extracted from the entire data, which may be in the form of structured or unstructured data. The ontology is incrementally and continuously refined after the initial creation by the interaction of the AI system with the real world as new data becomes available. In addition, the curated data is also used for prediction of the risk score of the trial and determination of risk based monitoring strategy for the trial.

As the model allows for understanding the various features, the model may determine the risk categories and the risk elements which need more weightage. Further, the model may determine the preference that may be given to certain training datasets, such as the site specific risk elements and the patient specific risk elements, for predicting the overall risk score of the clinical trial. This results in data explainability, which is understanding the data that is used as input and for training purposes, and how the end result of the model is predicted. In an embodiment, the model may allow the leadership team and researchers to interpret how it is arriving at the overall risk score for the clinical trial, and how that is used to determine the monitoring decisions relative to the associated risk. The model also provides for model explainability so that the user understands the model, how the end result is reached, thereby resulting in more acceptance of the model. Trust in the model allows the model to be easily accepted by the patients, caregivers, and regulatory agencies. Apart from data and model explainability, explainability may also be provided on the other two axes, namely, post hoc explainability and assessment of explanations. Post hoc explainability may solve the problem of opaqueness of the AI model by providing logic to the internal working of the model and making it easy to understand and trustworthy for the users. Some of the methods for handling post hoc explainability may include visualization methods, game theory methods, and neural methods. Visualization methods may be used to present the data of the clinical trial and it may aid in planning and forecasting the risk for the trial ahead of the trial starting. Assessment or evaluation of explanations of the AI model assesses the model on data explainability, model explainability, and post hoc explainability. Assessment provides for more user empowerment with regard to the AI model. As the actions of the model are explained by Explainability of AI, it results in more transparency and acceptance of the model by the users.

Decision trees may be one of the techniques used by the AI model for predictive modelling. It builds its decisions based on the input data and may be employed for classification and regression assignments. In decision tree, data may be represented on nodes, such as root node, internal node, branch node, and leaf node. In an embodiment, the root node may represent the risk categories and the risk elements may be defined by the internal node. The branch node may define the weightages associated with the risk categories and the risk elements. The risk score may be defined by the leaf node which represents the prediction of the overall risk score of the clinical trial. The decision making by the Decision Tree algorithm may be used for providing risk based monitoring of the clinical trial. By considering the planning datasets for various site specific risk factors and patient specific risk factors, decision trees may predict the risk score of the trial ahead of the trial starting, and enable to define risk based monitoring of the trial in advance.

In an embodiment, the AI model employed for ascertaining the risk based monitoring may use Bayesian Network. As the site and patient specific risk factors change over the course of a clinical trial, a Bayesian Network may be employed by the AI model for predicting the risk score of the trial. The Bayesian network uses probability theory for prediction and provides decision making under uncertain conditions such as the ones that may arise during the course of a clinical trial. Bayesian network uses nodes and arcs as in the decision trees algorithm, for providing predictive analytics and identifying patterns thereby enabling risk based monitoring decisions for the clinical trial. The Bayesian network may model the relationship between the various risk categories and risk elements, and the outcome in the form of risk score of the trial, to enable better management of risk based monitoring of the trial.

In an embodiment, the AI model may employ a sparse neural network for the assessment of risk based monitoring of the clinical trial. As this model focuses on the most relevant parameters and components, it provides for reduced computations and less memory usage while the efficiency is enhanced. By using this model, the risk factors that mainly affect the monitoring and influence the outcome may be considered for determination of risk score of the clinical trial and the type of monitoring to be used for the trial. Sparse model may use lasso technique for identification of the relevant features that can impact the outcome. Sparse model may also provide interpretability as the relevant features are selected from the entire dataset, in order to predict the risk score of the clinical trial.

Techniques such as dropout and regularization can be utilized to reduce the bias in the model's prediction and increase its capability to generalize knowledge from the various characteristics to predict an overall clinical trial risk. Further, the model's hyper parameters such as depth of the network, dimensions, learning rate and momentum can be fine-tuned to improve the power of predictability of risk by leveraging the optimization techniques including, but not limited to, gradient descent, stochastic gradient descent, and their flavors (speed, memory, noise).

Each model in the system computes an overall risk score for global, local, site specific and patient specific risks considered, to provide a naive measure that comprehensively summarizes the data monitoring to be carried out in the form of the overall clinical trial risk score. The system, in various embodiments of the claimed invention can then compute the overall risk score using statistical techniques that derive the score from each of the above models (learning models, logistic regression models, neural networks, SVM, CNN models, RNN models, LSTM models).

Risk Stratification and Insight Delivery: To increase the viability of clinical trial risk score interpretation, the prediction results can be stratified into Low, Medium, and High. This is done by the system by employing modules of statistical techniques to perform operations such as normalization, standardization of predicted values and identification of thresholds to classify a risk score as low, medium, and high. Such a classification of risk score, in various embodiments of the claimed invention, can help in easy assessment and interpretation of the overall risk score of the clinical trial.

Feed-back layer: In addition, the system also provides its algorithms the self-learning capabilities to learn continuously from the data provided. Such an ability in various embodiments of the claimed invention can be potentially useful in identifying and designing optimal risk based monitoring.

The system includes in various embodiments of the claimed invention an AI suite which executes multiple machine learning models to find the optimum model yielding highest metrics of evaluation. The AI suite includes, but is not limited to, models as simple as logistic regression, and Support Vector Machine (SVM) regression to complex models such as neural networks including, but is not limited to, convolutional neural network (CNN), recurrent neural network (RNN) and long short-term memory model (LSTM).

In one possible configuration of the system, all available types of input data can be used to train multiple models and the best model will be employed for predicting the risk score related to the clinical trial risk based monitoring.

In another possible configuration of the system, multiple machine learning models will be trained on a subset of data and the best ensemble of those models will be employed for the prediction in various embodiments of the claimed invention. For example, CNN models will be trained using image scans data, RNN models will be trained using clinical notes and medical history data and so on. Then best performing models from each input data type will be assessed for concordance among them and then all those models will be ensembled or stacked together to predict the risk score for the clinical trial.

The system not only uses the structured data fields like age, race, but also uses unstructured data from sources like patient diary, patient reports, audio, and video files of patient encounters. It uses a comprehensive list of data fields which include demographic information, clinical data (e.g., patient history), routine laboratory tests, investigational biomarkers, genetic testing, microbiome, imaging studies (esp. ultrasound), medications, clinical notes—by physicians, nurses, site staff, nutritional data, patient experience scores, institutional data—to investigate the impact of practice patterns (e.g., Site maturity profile), physician data (for examining the impact of individual providers on outcome) and audio/video files of Clinician-patient Interactions.

The models leverage advanced computing capabilities and are not limited to: Artificial Intelligence (including neural networks, Natural Language Processing and understanding, deep learning) and traditional statistical techniques and can analyse structured and unstructured data sets including, but not limited to: biomarkers and biochemistry data, images, genetics, clinician notes, audios and videos, demographic and socio-economic data.

The system continuously receives real-time feed-back from caregivers and site staff and improvises the risk score on a perpetual basis. It leverages cutting-edge computing capabilities of AI, Mathematics and Statistics, analyses relevant data (e.g., genetics, images, clinician's notes, audio and videos, healthcare records, wearable devices, pathology etc.) and generates unparalleled insights about diseases, their evolution, and the impact of interventions.

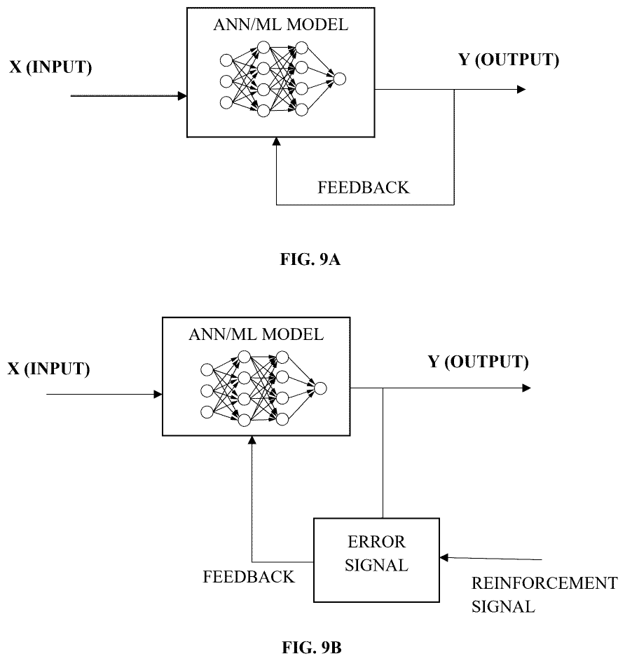

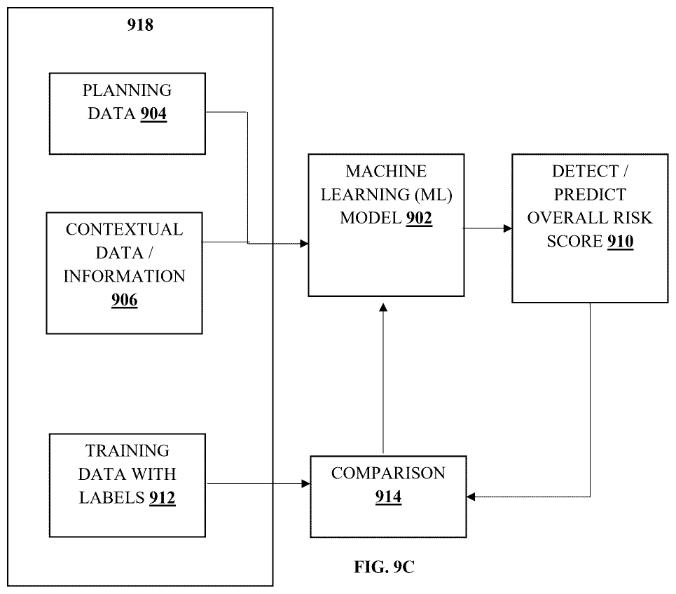

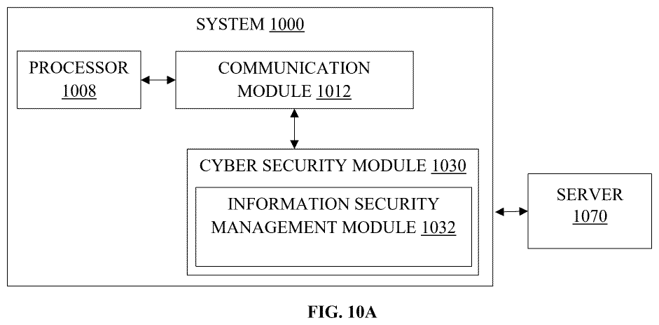

FIG. 9A shows a structure of the neural network/machine learning model with a feedback loop. An Artificial neural networks (ANNs) model comprises an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed to the next layer of the network. A machine learning model or an ANN model may be trained on a set of data to take a request in the form of input data, make a prediction on that input data, and then provide a response. The input to the model may include planning and forecasting risk elements for the site and patient specific risk categories. Planning data sets for site recruitment and patient recruitment for the clinical trial sites may be used as input, in order to make a prediction of the overall risk score of the clinical trial. This helps in determining the level of monitoring and the type of monitoring required for each of the clinical trial sites. The model may learn from the data. Learning can be supervised learning and/or unsupervised learning and may be based on different scenarios and with different datasets. Supervised learning comprises logic using at least one of a decision tree, logistic regression, and support vector machines. Unsupervised learning comprises logic using at least one of a k-means clustering, a hierarchical clustering, a hidden Markov model, and an apriori algorithm. The output layer may predict or detect the overall risk score for the clinical trial based on a predefined threshold value of the overall risk score. In an embodiment, risk elements related to site maturity profile may be used as input data. This includes the number of studies completed at the site, studies in a specific therapeutic area, patient throughput, the number of years the site has been functional, site relationships to other health campuses locally, staff churn, staff experience, etc. These details are fed into the ANN/ML model to obtain an output. The output layer may predict or detect the overall risk score for the clinical trial site based on a predefined threshold value of the overall risk score. The model may predict that with higher maturity profile of the site, less monitoring may be needed. Based on the overall risk score, the level, and the type of monitoring of the clinical trial site may be determined. In another embodiment, risk elements related to site experience profile may be used as input data. Site experience profile may include the level of staff expertise in the country/area, in the specific sites under consideration, and the experience of clinical trial staff and caregivers available for the specific therapeutic area for the clinical trial. The output layer may predict the overall risk score for the clinical trial site, and further predict that when the site experience profile is high, there is less need for site monitoring and the model predicts that the site may begin at 50% remote SDV.

In an embodiment, ANNs may be a Deep-Neural Network (DNN), which is a multilayer tandem neural network comprising Artificial Neural Networks (ANN), Convolution Neural Networks (CNN) and Recurrent Neural Networks (RNN) that can recognize features from inputs, do an expert review, and perform actions that require predictions, creative thinking, and analytics. In an embodiment, ANNs may be Recurrent Neural Network (RNN), which is a type of Artificial Neural Networks (ANN), which uses sequential data or time series data. Deep learning algorithms are commonly used for ordinal or temporal problems, such as language translation, Natural Language Processing (NLP), speech recognition, and image recognition, etc. Like feedforward and convolutional neural networks (CNNs), recurrent neural networks utilize training data to learn. They are distinguished by their “memory” as they take information from prior input via a feedback loop to influence the current input and output. An output from the output layer in a neural network model is fed back to the model through the feedback. The variations of weights in the hidden layer(s) will be adjusted to fit the expected outputs better while training the model. This will allow the model to provide results with far fewer mistakes.

The neural network is featured with the feedback loop to adjust the system output dynamically as it learns from the new data. In machine learning, backpropagation and feedback loops are used to train an AI model and continuously improve it upon usage. As the incoming data that the model receives increases, there are more opportunities for the model to learn from the data. The feedback loops, or backpropagation algorithms, identify inconsistencies and feed the corrected information back into the model as an input.

Even though the AI/ML model is trained well, with large sets of labelled data and concepts, after a while, the models' performance may decline while adding new, unlabelled input due to many reasons which include, but not limited to, concept drift, recall precision degradation due to drifting away from true positives, and data drift over time. A feedback loop to the model keeps the AI results accurate and ensures that the model maintains its performance and improvement, even when new unlabelled data is assimilated. A feedback loop refers to the process by which an AI model's predicted output is reused to train new versions of the model.

Initially, when the AI/ML model is trained, a few labelled samples comprising both positive and negative examples of the concepts (e.g., metadata on risk categories and risk elements) are used that are meant for the model to learn. This may include planning data sets for site recruitment and patient recruitment. Afterward, the model is tested using unlabelled data. By using, for example, deep learning and neural networks, the model can then make predictions on whether the desired concept/s (e.g., the overall risk score of the clinical trial) are in unlabelled images. Each image is given a probability score where higher scores represent a higher level of confidence in the models' predictions. Where a model gives an image a high probability score, it is auto-labelled with the predicted concept. However, in the cases where the model returns a low probability score, this input may be sent to a controller (may be a human moderator) which verifies and, as necessary, corrects the result. The human moderator may be used only in exception cases. The feedback loop feeds labelled data, auto-labelled or controller-verified data back to the model dynamically and is used as training data so that the system can improve its predictions in real-time and dynamically.

FIG. 9B shows a structure of the neural network/machine learning model with reinforcement learning. The network receives feedback from authorized networked environments. Though the system is similar to supervised learning, the feedback obtained in this case is evaluative not instructive, which means there is no teacher as in supervised learning. After receiving the feedback, the network performs adjustments of the weights to get better predictions in the future. Machine learning techniques, like deep learning, allow models to take labelled training data and learn to recognize those concepts in subsequent data and images. The model may be fed with new data for testing, hence by feeding the model with data it has already predicted over, the training gets reinforced. If the machine learning model has a feedback loop, the learning is further reinforced with a reward for each true positive of the output of the system. Feedback loops ensure that AI results do not stagnate. By incorporating a feedback loop, the model output keeps improving dynamically and over usage/time.

FIG. 9C shows an example block diagram for predicting the overall risk score of a clinical trial using a machine learning model. The machine learning model 902 may take as input data planning sets for site recruitment and patient recruitment and learn to identify associated metadata and parameters within the data that are predictive of feasibility and selection of clinical trial sites. The training data sample may include, for example, planning data 904, which may be the metadata associated with site recruitment, such as site feasibility threshold, ease of patient access, investigator experience, staff availability and experience, key opinion leadership, number of studies performed, and ranking of the site relative to other sites in the trial, etc. Further, the site specific risk elements and patient specific risk elements may be provided in real-time. The site specific risk elements may include site feasibility, prior history of the site, site permanency, site selection and site recruitment plan. Site feasibility may include how able is the site and how far ahead it is of the feasibility threshold. Example elements include equipment availability, ease of patient access, investigator experience, staff availability and experience, key leadership, etc. Prior history of the site may include number of studies performed, relative ranking in terms of execution to plan (patient recruitment, deviations, error rates, late/incomplete data submission, response times). Site permanency may include: is the site a popup site versus one in place and aligned to a health facility for an extended period of time and also involvement with local communities of care. Site selection may include a measure of the contract negotiation process and how onerous/difficult it was with the site, and it may be an indicator of the future relationships with the site. Site recruitment plan may include ranking of the site relative to other sites in the study and determining whether to recruit the site for the clinical trial. The patient specific risk elements may include patient recruitment planning, prior history of the patient, patient selection criteria, and patient recruitment forecast. The patient recruitment planning may include a more/less aggressive patient recruitment plan than prior studies based on therapeutic area, inclusion/exclusion criteria, protocol complexity, etc. Prior history of the patient may include involvement of the patient in other trials and experience of what it takes to engage to the end of the trial. Patient selection criteria may include complexity of the selection process and, based on the criteria (age, location, etc.), how likely is the patient to engage given that they are selected. Patient recruitment forecast may include the forecast compared to the feasibility assessment and how the site can support the forecast in terms of staff, facilities, equipment, access, etc.

In an embodiment, the training data sample may also include current contextual information 906 relating to the clinical trial. This may include, for example site specific risk elements and patient specific risk elements provided in real-time. For example, contextual information 906 for site specific risk elements may include the geographic area of the site, socio economic profile of the area, site maturity profile, site regulatory environment, and site experience profile. The geographic area of the site may be at a location with civil/military unrest, political chum, and may indirectly make it difficult to execute the trial, and potentially limit access to the site. The socio economic profile of the area may include relative ranking on area wealth, education, crime, employment rates, transport infrastructure, communications infrastructure, health services, which are specific to the particular site and the demographics and medical characteristics of the patient population served by the site, including age, gender, ethnicity, and relevant medical conditions. The site maturity profile may include factors such as number of studies completed, studies completed in a specific therapeutic area, patient throughput, years the site has been functional, site relationships to other health campuses locally, staff chum, staff experience, etc. The site regulatory environment may include the regulatory framework governing clinical research at the site's location, including ethics review boards, regulatory agencies, and compliance with local laws and guidelines. The site experience profile may include the level of expertise in the area, and the specific sites under consideration, the expertise would be the experience of clinical trial staff such as principal investigators, sub-investigators, and study coordinators and caregivers available for a site for a particular therapeutic area for which the clinical trial is to be conducted. These contextual details help researchers, sponsors, and regulatory authorities evaluate the suitability and readiness of a clinical trial site for conducting research effectively and ethically.

The contextual information 906 for patient specific risk elements may include prior history of the patient, medical history of the patient, patient lifestyle factors, psychosocial factors, patient family history and comorbidities. Prior history of the patient may include enrollment of the patient in previous clinical trials and the engagement of the patient until the end of the trial. It may also include whether the patient submitted erroneous data in the past. The medical history of the patient may include previous illnesses, surgeries, and chronic conditions, which can provide insights into how underlying health issues might influence treatment outcomes, including potential genetic markers that might affect treatment efficacy or side effects. Patient lifestyle factors may include diet, exercise habits, smoking status, alcohol consumption, and environmental exposures, that can impact treatment outcomes. Psychosocial factors may include factors such as stress, mental health conditions, social support networks, and quality of life which can influence how patients respond to treatment and their overall well-being during the trial. Patient family history and comorbidities may include genetic predispositions and potential hereditary conditions that may impact treatment effectiveness and other medical conditions that a patient may have in addition to the condition being studied in the clinical trial that can impact treatment outcomes and help researchers assess the generalizability of study results to patients with multiple health issues.

Any of the aforementioned types of data (e.g., planning data 904, contextual information 906, or any other data) may correlate with the prediction of an overall risk score of the clinical trial, and such correlation may be automatically learned by the machine learning model 902. In an embodiment, during training, the machine learning model 902 may process the training data sample (e.g., planning data 904 and/or contextual information 906) and, based on the current parameters of the machine learning model 902, detect or predict the overall risk score of the clinical trial 910. The detection or prediction of the overall risk score 910 may depend on the training data with labels 912 associated with the training sample 918. Predicting an overall risk score refers to predicting a future event based on past and present data and most commonly by analysis of trends or data patterns. Prediction or predictive analysis employs probability based on the data analyses and processing. Predicted events may or may not require changes in monitoring based on how the turn of events happen. For example, the system based on local weather, or an environmental event may predict a disruption in monitoring of clinical trial data for a particular site, but it may not turn into a change in monitoring approach as the condition may be short lived to have any impact on the monitoring of the clinical trial site. However, if the condition does not improve, then the system detects that it is an issue that may cause a change in monitoring approach. For example, if the training label 912 indicates the particular site condition the machine learning model 902 would learn to detect the issue based on input data associated with a given planning data 904 and contextual information 906. This results in improving the model during the clinical trial. The model may be tested periodically at different intervals of time, such as when new training data is added, or when the new training data is different from the training data. In an embodiment, during training, the detected or predicted risk score at 910 and the training data with labels 912 may be compared at 914. For example, the comparison at 914 may be based on a loss function that measures a difference between the detected or predicted risk score 910 and the training data with labels 912. Based on the comparison at 914 or the corresponding output of the loss function, a training algorithm may update the parameters of the machine learning model 902, with the objective of minimizing the differences or loss between subsequent detections or predictions of the risk score 910 and the corresponding labels 912. By iteratively training in this manner, the machine learning model 902 may “learn” from the different training data samples and become better at detecting or predicting the clinical trial risk score at 910 that are similar to the ones represented by the training labels at 912.

In an embodiment, planning data 904 from the clinical trial site planning dataset or the patient planning dataset that is associated with a time segment may be labelled 912 or associated with one or more predetermined event types to represent what transpired or occurred with regard to the monitoring of the clinical trial during that time segment. For example, a particular training data sample 918 capturing prior history, involvement, and engagement experience of a patient in other clinical trials and they may be labelled 912 or associated with a “patient prior history” event type. For example, a particular training data sample 918 may pertain to a particular incident that occurred in the past. The training data sample 918 may be labelled 912 or associated with the “patient prior history” event type, and the planning data 904 may include further data of the event such as patient selection criteria, along with contextual information 906, such as patient age, location, etc.

Using the training data, a machine learning model 902 may be trained so that it recognizes features of input data that signify or correlate to certain event types. For example, a trained machine learning model 902 may recognize data features that signify the likelihood of a rising risk situation, as an actionable event. In an embodiment, the features may have meaningful interpretations, such as, a site is proving to be a rising risk due to timeliness or quality of data being received, or if patients at a specific site are showing a higher risk of abandoning the trial, or if the site is temporary or multi-functional in nature, etc. In an embodiment, the events that change the risk rating are used as training data or input data for training. The features result in improvement of the model as the clinical trial progresses. For example, if a site is temporary or multi-functional in nature, such as, a mobile van might park at a supermarket to provide a basic clinical trial site, or the site might also be a pharmacy or other local care centre may be used for indicating an increase in monitoring for that site. The features may also be unintelligible to humans and may simply represent data patterns that tend to be present when certain event types occur. Through training, the machine learning model 902 may learn to identify predictive and non-predictive features and apply the appropriate weights to the features to optimize the machine learning model's 902 predictive accuracy. In embodiments where supervised learning is used and each training data sample 918 has a training data with labels 912, the training algorithm may iteratively process each training data sample 918 (including planning data 904 and/or contextual information 906) and generate a prediction 910 based on the model's 902 current parameters. Based on the comparison results at 914, the training algorithm may adjust the model's 902 parameters/configurations (e.g., weights) accordingly to minimize the differences between the generated predictions 910 and the corresponding labels 912. Any suitable machine learning model and training algorithm may be used, including, e.g., neural networks, decision trees, clustering algorithms, and any other suitable machine learning techniques. Once trained, the machine learning model 902 may take input data associated with a site and output one or more predictions that indicate a likelihood that when the site may be recruited into the study, which will help predict risk ahead of the trial starting. In an embodiment, the input data, metadata associated with a site or a patient may be different from any of the metadata associated with the training data samples. In an embodiment, it relates to systems and methods that predict an overall risk score for a clinical trial to determine the level of monitoring required for a site. The systems and methods of the present disclosure may also provide data analytics information that may be used later to improve the clinical trial risk score prediction.

In an embodiment, the system may comprise a cyber security module.

In one aspect, a secure communication management (SCM) computer device for providing secure data connections is provided. The SCM computer device includes a processor in communication with memory. The processor is programmed to receive, from a first device, a first data message. The first data message is in a standardized data format. The processor is also programmed to analyse the first data message for potential cyber security threats.

According to an embodiment, secure authentication for data transmissions comprises, provisioning a hardware-based security engine (HSE) located in communications system, said HSE having been manufactured in a secure environment and certified in said secure environment as part of an approved network; performing asynchronous authentication, validation and encryption of data using said HSE, storing user permissions data and connection status data in an access control list used to define allowable data communications paths of said approved network, enabling communications of the communications system with other computing system subjects to said access control list, performing asynchronous validation and encryption of data using security engine including identifying a user device (UD) that incorporates credentials embodied in hardware using a hardware-based module provisioned with one or more security aspects for securing the system, wherein security aspects comprising said hardware-based module communicating with a user of said user device and said HSE.

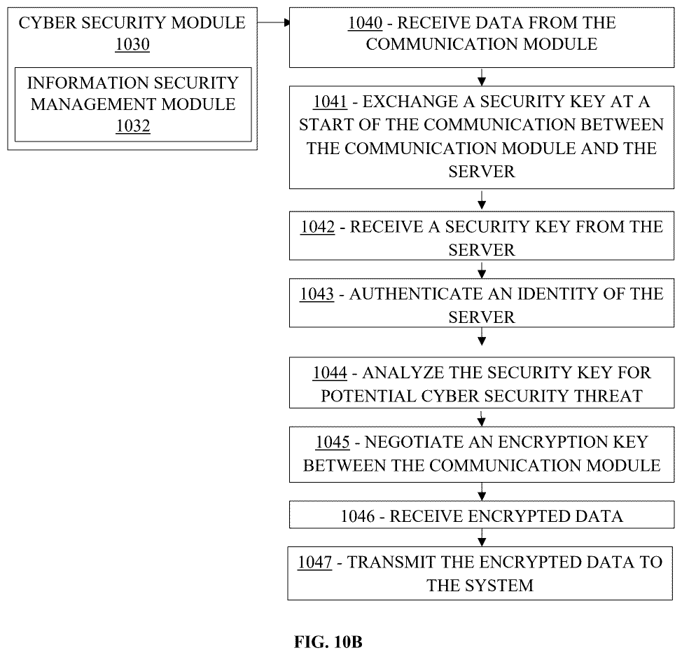

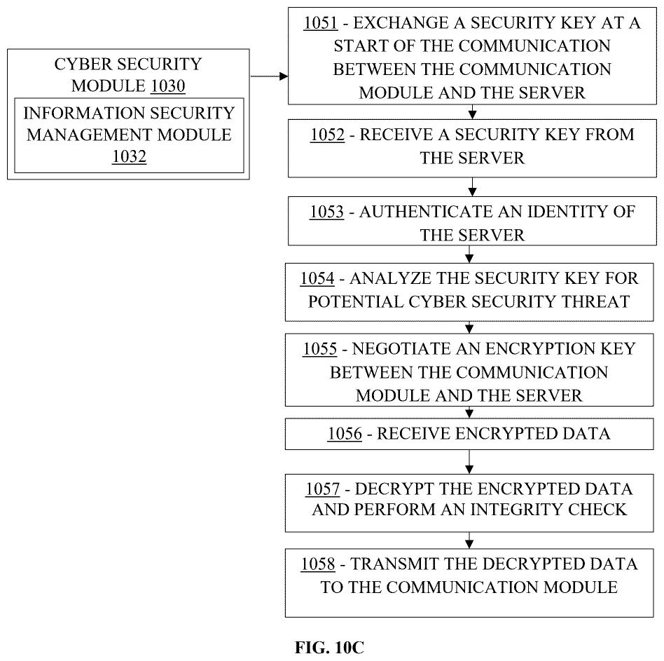

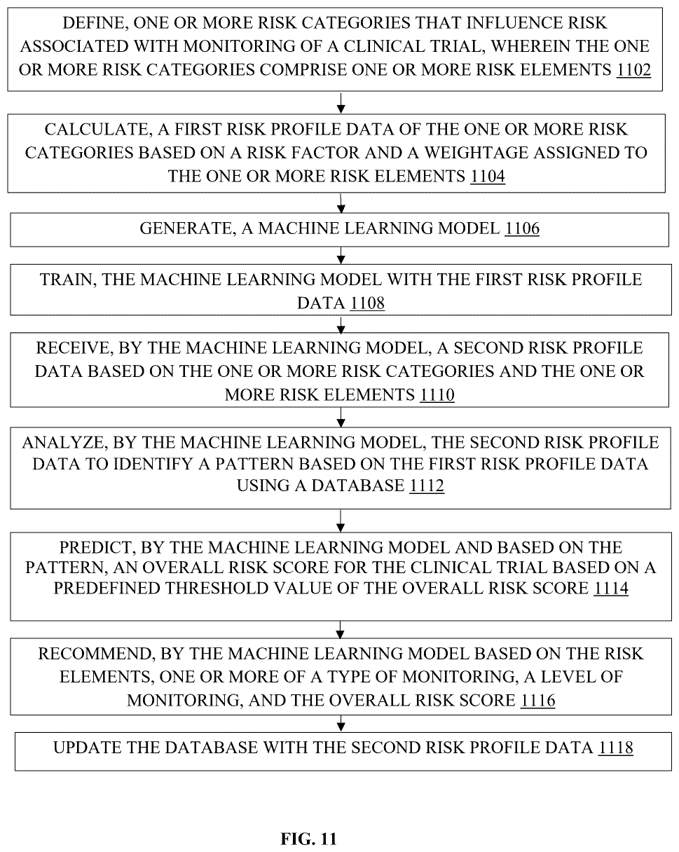

In an embodiment, FIG. 10A shows the block diagram of the cyber security module. The communication of data from the processor 1008 of the system 1000 and the server 1070 through the communication module 1012 is first verified by the information security management module 1032 before being transmitted from the system to the server or from the server to the system. The information security management module is operable to analyse the data for potential cyber security threats, to encrypt the data when no cyber security threat is detected, and to transmit the data encrypted to the system or the server In an embodiment, the communication of data from the communication devices 102 to the server 112 in FIG. 1, may be the data that is communicated by the communication module 1012 to the server 1070. The communication of data from the communication devices 102 to the database 114, the analytics communication device 116, the predictive communication device 118, the administrator communication device 120, and with other communication devices 122 in FIG. 1, may be first verified by the information security management module 1032 of the cyber security module 1030 to ensure integrity of data in the clinical trial. Once the data is gathered by the communication devices 102, for that data to accurately help determine the risk score of the site, or the patient, or the clinical trial, it may be kept from unauthorized access by verification provided by the cyber security module 1030. In an embodiment, the data transferred from medical devices communicating data to the communication devices 102 may also be verified by the cyber security module to ensure that the medical devices or wearable medical devices and corresponding apps have not been tampered with and the patient health is not put at risk. Further, as clinical trials include data relating to participants and patients, to ensure that the data is not compromised, the privacy and security of data may be protected by verifying the data by the cyber security module 1030.

The wearable medical devices may comprise one or more of a headband, such as an optical head-mounted display (OHMD), a smart watch, an adhesive patch, wearable clothing, camera clips, wearable biochemical sensors, RFID Tag, Pedal system, graphene electronic tattoo (GET), mood tracking applications, wearables with Inertial Measurement Unit (IMU) sensors, wearable data gloves, etc. Wearable devices can prompt positive behaviour, remind patients of necessary actions, such as take proper medication, and at the right time and provide immediate feedback, thus reducing the incidence of missing data, which is a significant challenge in clinical trials. Continuous monitoring, through wearable devices may enable early detection of adverse events or significant health changes, allowing for timely interventions. The wearable devices may provide real-time monitoring of the patient and their vitals during the trial. The wearable device may also serve as a safety alert device with fall detection and emergency calling features. The wearable device may also passively collect data such as patient's positional data, vital sign data, and physiological data, in real-time. This technology enables increased volume and speed of data collection, as compared to manual methods in traditional clinical trials, leading to further reduced data acquisition costs. This also helps monitor patients' physical and emotional health in real-time outside of the clinic and could help reduce expensive site visits by the patient.

In an embodiment, the cyber security module further comprises an information security management module providing isolation between the system and the server. FIG. 10B shows the flowchart of securing the data through the cyber security module 1030. At step 1040, the information security management module is operable to receive data from the communication module. At step 1041, the information security management module exchanges a security key at a start of the communication between the communication module and the server. At step 1042, the information security management module receives a security key from the server. At step 1043, the information security management module authenticates an identity of the server by verifying the security key. At step 1044, the information security management module analyses the security key for potential cyber security threats. At step 1045, the information security management module negotiates an encryption key between the communication module and the server. At step 1046, the information security management module receives the encrypted data. At step 1047, the information security management module transmits the encrypted data to the server when no cyber security threat is detected.

In an embodiment, FIG. 10C shows the flowchart of securing the data through the cyber security module 1030. At step 1051, the information security management module is operable to: exchange a security key at a start of the communication between the communication module and the server. At step 1052, the information security management module receives a security key from the server. At step 1053, the information security management module authenticates an identity of the server by verifying the security key. At step 1054, the information security management module analyses the security key for potential cyber security threats. At step 1055, the information security management module negotiates an encryption key between the communication module and the server. At step 1056, the information security management module receives encrypted data. At step 1057, the information security management module decrypts the encrypted data, and performs an integrity check of the decrypted data. At step 1058, the information security management module transmits the decrypted data to the communication module when no cyber security threat is detected.

In an embodiment, the integrity check is a hash-signature verification using a Secure Hash Algorithm 256 (SHA256) or a similar method.

In an embodiment, the information security management module is configured to perform asynchronous authentication and validation of the communication between the communication module and the server.

In an embodiment, the information security management module is configured to raise an alarm if a cyber security threat is detected. In an embodiment, the information security management module is configured to discard the encrypted data received if the integrity check of the encrypted data fails.

In an embodiment, the information security management module is configured to check the integrity of the decrypted data by checking accuracy, consistency, and any possible data loss during the communication through the communication module.

In an embodiment, the server is physically isolated from the system through the information security management module. When the system communicates with the server as shown in FIG. 10A, identity authentication is first carried out on the system and the server. The system is responsible for communicating/exchanging a public key of the system and a signature of the public key with the server. The public key of the system and the signature of the public key are sent to the information security management module. The information security management module decrypts the signature and verifies whether the decrypted public key is consistent with the received original public key or not. If the decrypted public key is verified, the identity authentication is passed. Similarly, the system and the server carry out identity authentication on the information security management module. After the identity authentication is passed on to the information security management module, the two communication parties, the system, and the server, negotiate an encryption key and an integrity check key for data communication of the two communication parties through the authenticated asymmetric key. A session ID number is transmitted in the identity authentication process, so that the key needs to be bound with the session ID number; when the system sends data to the outside, the information security gateway receives the data through the communication module, performs integrity authentication on the data, then encrypts the data through a negotiated secret key, and finally transmits the data to the server through the communication module. When the information security management module receives data through the communication module, the data is decrypted first, integrity verification is carried out on the data after decryption, and if verification is passed, the data is sent out through the communication module; otherwise, the data is discarded.

In an embodiment, the identity authentication is realized by adopting an asymmetric key with a signature.

In an embodiment, the signature is realized by a pair of asymmetric keys which are trusted by the information security management module and the system, wherein the private key is used for signing the identities of the two communication parties, and the public key is used for verifying that the identities of the two communication parties are signed. Signing identity comprises a public and a private key pair. In other words, signing identity is referred to as the common name of the certificates which are installed in the user's machine.

In an embodiment, both communication parties need to authenticate their own identities through a pair of asymmetric keys, and a task in charge of communication with the information security management module of the system is identified by a unique pair of asymmetric keys.

In an embodiment, the dynamic negotiation key is encrypted by adopting an Rivest-Shamir-Adleman (RSA) encryption algorithm. RSA is a public-key cryptosystem that is widely used for secure data transmission. The negotiated keys include a data encryption key and a data integrity check key.

In an embodiment, the data encryption method is a Triple Data Encryption Algorithm (3DES) encryption algorithm. The integrity check algorithm is a Hash-based Message Authentication Code (HMAC-MD5-128) algorithm. When data is output, the integrity check calculation is carried out on the data, the calculated Message Authentication Code (MAC) value is added with the header of the value data message, then the data (including the MAC of the header) is encrypted by using a 3DES algorithm, the header information of a security layer is added after the data is encrypted, and then the data is sent to the next layer for processing. In an embodiment the next layer refers to a transport layer in the Transmission Control Protocol/Internet Protocol (TCP/IP) model.

The information security management module ensures the safety, reliability, and confidentiality of the communication between the system and the server through the identity authentication when the communication between the two communication parties starts the data encryption and the data integrity authentication. The method is particularly suitable for an embedded platform which has less resources and is not connected with a Public Key Infrastructure (PKI) system and can ensure that the safety of the data on the server cannot be compromised by a hacker attack under the condition of the Internet by ensuring the safety and reliability of the communication between the system and the server.

FIG. 11 shows a flow diagram for a method for enabling risk based monitoring (RBM) in a clinical trial according to one or more embodiments. Disclosed is a method comprising defining, one or more risk categories that influence risk associated with monitoring of a clinical trial, wherein the one or more risk categories comprise one or more risk elements at step 1102; calculating, a first risk profile data of the one or more risk categories based on a risk factor and a weighting assigned to the one or more risk elements at step 1104; generating, a machine learning model at step 1106; training, the machine learning model with the first risk profile data at step 1108; receiving, by the machine learning model, a second risk profile data based on the one or more risk categories and the one or more risk elements at step 1110; analysing, by the machine learning model, the second risk profile data to identify a pattern based on the first risk profile data using a database at step 1112; predicting, by the machine learning model and based on the pattern, an overall risk score for the clinical trial based on a predefined threshold value of the overall risk score at step 1114; recommending, by the machine learning model based on the risk elements, one or more of a type of monitoring, a level of monitoring, and the overall risk score at step 1116; updating the database with the second risk profile data at step 1118; and wherein the machine learning model is a self-learning model comprising a feed-back layer that enables the machine learning model to learn continuously from the second risk profile data and improve the prediction of the overall risk score and monitoring decisions to enable risk based monitoring (RBM) of the clinical trial.

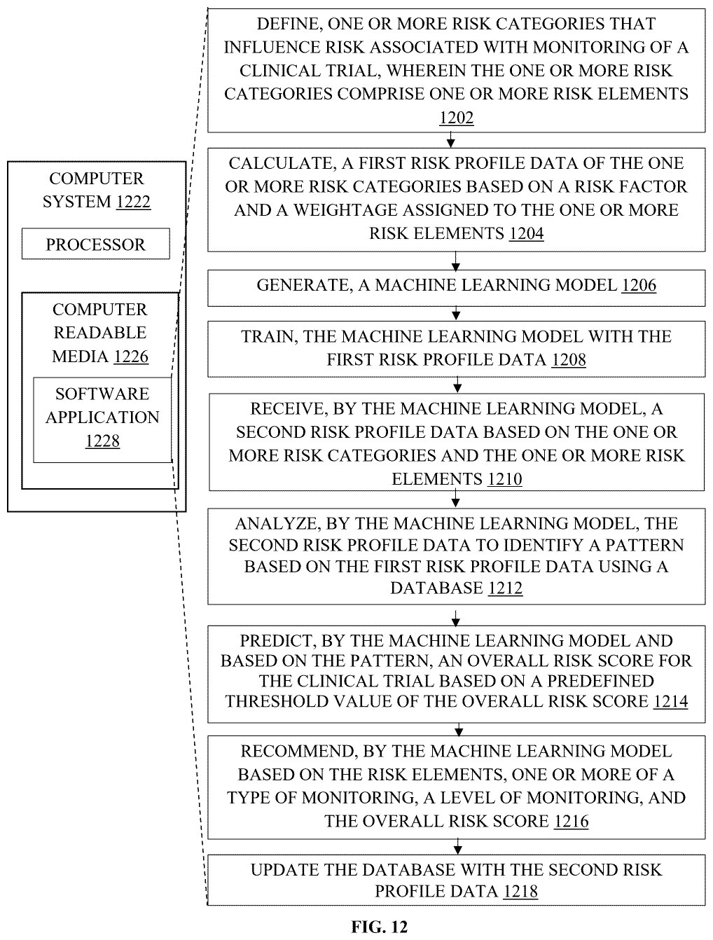

FIG. 12 shows a flow diagram for a non-transitory computer-readable medium 1226 for enabling risk based monitoring (RBM) in a clinical trial according to one or more embodiments. According to an embodiment, disclosed is a non-transitory computer-readable medium 1226, comprising software application 1228, having stored thereon instructions executable by a computer system 1222 comprising a processor 1224 to perform operations comprising, defining, one or more risk categories that influence risk associated with monitoring of a clinical trial, wherein the one or more risk categories comprise one or more risk elements at step 1202; calculating, a first risk profile data of the one or more risk categories based on a risk factor and a weighting assigned to the one or more risk elements at step 1204; generating, a machine learning model at step 1206; training, the machine learning model with the first risk profile data at step 1208; receiving, by the machine learning model, a second risk profile data based on the one or more risk categories and the one or more risk elements at step 1210; analysing, by the machine learning model, the second risk profile data to identify a pattern based on the first risk profile data using a database at step 1212; predicting, by the machine learning model and based on the pattern, an overall risk score for the clinical trial based on a predefined threshold value of the overall risk score at step 1214; recommending, by the machine learning model based on the one or more risk elements, a type of monitoring, a level of monitoring, and the overall risk score at step 1216; updating the database with the second risk profile data at step 1218; and wherein the machine learning model is a self-learning model comprising a feed-back layer that enables the machine learning model to learn continuously from the second risk profile data and improve the prediction of the overall risk score and monitoring decisions to enable risk based monitoring (RBM) of the clinical trial.

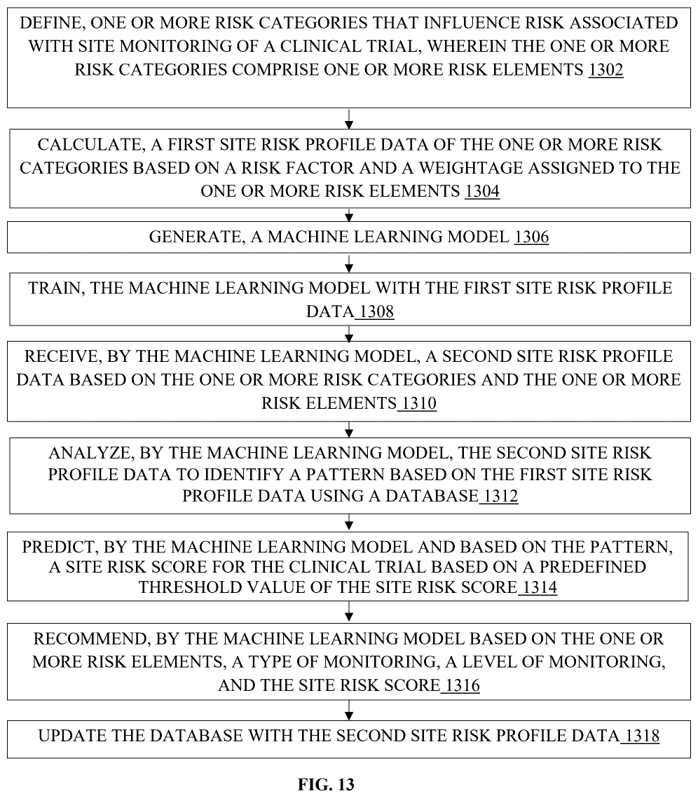

FIG. 13 shows a flow diagram for a method for enabling risk based monitoring (RBM) of a site in a clinical trial according to one or more embodiments. According to an embodiment, disclosed is a method comprising, defining, one or more risk categories that influence risk associated with site monitoring of a clinical trial, wherein the one or more risk categories comprise one or more risk elements at step 1302; calculating, a first site risk profile data of the one or more risk categories based on a risk factor and a weighting assigned to the one or more risk elements at step 1304; generating, a machine learning model at step 1306; training, the machine learning model with the first site risk profile data at step 1308; receiving, by the machine learning model, a second site risk profile data based on the one or more risk categories and the one or more risk elements at step 1310; analysing, by the machine learning model, the second site risk profile data to identify a pattern based on the first site risk profile data using a database at step 1312; predicting, by the machine learning model and based on the pattern, a site risk score for the clinical trial based on a predefined threshold value of the site risk score at step 1314; recommending, by the machine learning model based on the one or more risk elements, a type of monitoring, a level of monitoring, and the site risk score at step 1316; updating the database with the second site risk profile data at step 1318; and wherein the machine learning model is a self-leaming model comprising a feed-back layer that enables the machine learning model to learn continuously from the second site risk profile data and improve the prediction of the site risk score and monitoring decisions to enable risk based monitoring (RBM) of a site in the clinical trial.

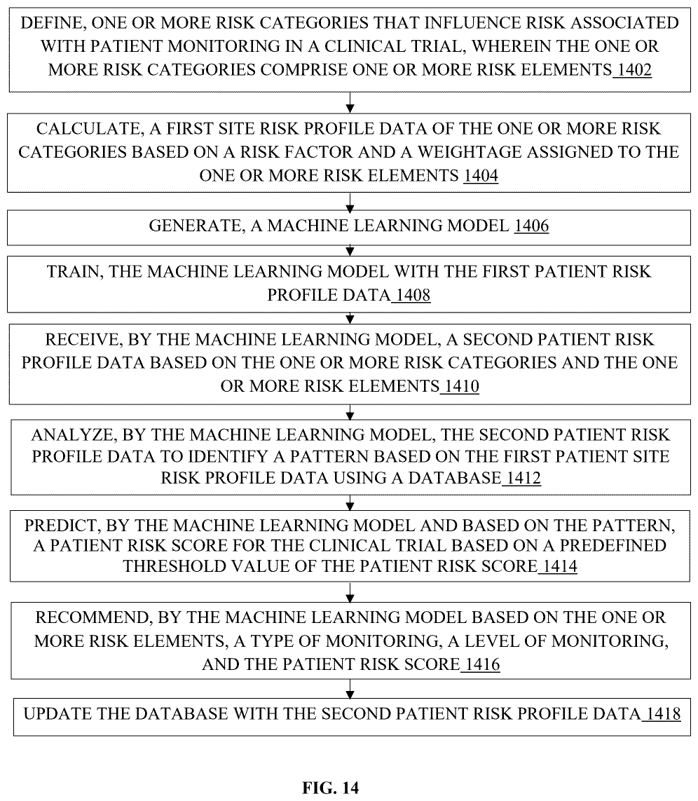

FIG. 14 shows a flow diagram for a method for enabling risk based monitoring (RBM) of a patient in a clinical trial according to one or more embodiments. According to an embodiment, disclosed is a method comprising, defining, one or more risk categories that influence risk associated with patient monitoring in a clinical trial, wherein the one or more risk categories comprise one or more risk elements at step 1402; calculating, a first patient risk profile data of the one or more risk categories based on a risk factor and a weighting assigned to the one or more risk elements at step 1404; generating, a machine learning model at step 1406; training, the machine learning model with the first patient risk profile data at step 1408; receiving, by the machine learning model, a second patient risk profile data based on the one or more risk categories and the one or more risk elements at step 1410; analysing, by the machine learning model, the second patient risk profile data to identify a pattern based on the patient risk profile data using a database at step 1412; predicting, by the machine learning model and based on the pattern, a patient risk score for the clinical trial based on a predefined threshold value of the patient risk score at step 1414; recommending, by the machine learning model based on the one or more risk elements, a type of monitoring, a level of monitoring, and the patient risk score at step 1416; updating the database with the second patient risk profile data at step 1418; and wherein the machine learning model is a self-learning model comprising a feed-back layer that enables the machine learning model to learn continuously from the second patient risk profile data and improve the prediction of the patient risk score and monitoring decisions to enable risk based monitoring (RBM) of patient in the clinical trial.

According to an embodiment, disclosed is a system comprising a processor storing instructions in a non-transitory memory that, when executed, cause the processor to define, one or more risk categories that influence risk associated with monitoring of a clinical trial, wherein the risk categories comprise one or more risk elements; calculate, a first risk profile data of the risk categories based on a risk factor and a weighting assigned to the risk elements; generate, a machine learning model; train, the machine learning model with the first risk profile data; receive, by the machine learning model, a second risk profile data based on the risk categories and the risk elements; analyse, by the machine learning model, the second risk profile data to identify a pattern based on the first risk profile data using a database; predict, by the machine learning model and based on the pattern, an overall risk score for the clinical trial based on a predefined threshold value of the overall risk score; recommend, by the machine learning model based on the risk elements, one or more of a type of monitoring, a level of monitoring, and the overall risk score; update the database with the second risk profile data; and wherein the machine learning model is a self-learning model comprising a feed-back layer that enables the machine learning model to learn from the second risk profile data and improve the prediction of the overall risk score and monitoring decisions to enable risk based monitoring (RBM) of the clinical trial.

According to an embodiment of the system, the risk categories comprise global risk category, local risk category, site specific risk category and patient specific risk category.

According to an embodiment of the system, the risk elements are defined for each of the global risk category, the local risk category, the site specific risk category and the patient specific risk category.

According to an embodiment of the system, the risk elements for the global risk category comprises global risk elements, the risk elements for the local risk category comprises local risk elements, the risk elements for the site specific risk category comprises site specific risk elements, and the risk elements for the patient specific risk category comprises patient specific risk elements.

According to an embodiment of the system, the global risk elements comprises one or more of a therapeutic area, a study phase, a protocol complexity, an interventional risk and an observational risk.

According to an embodiment of the system, the local risk elements comprises one or more of a geographic area, a socio economic profile, a site maturity profile, and a site experience profile.

According to an embodiment of the system, the site specific risk elements comprises one or more of a site feasibility, a prior history of a site, a site permanency, a site selection, and a site recruitment plan.

According to an embodiment of the system, the patient specific risk elements comprises one or more of a patient recruitment plan, a prior history of a patient, a patient selection criteria, and a patient recruitment forecast.

According to an embodiment of the system, the predefined threshold value of the overall risk score is initially set based on at least one of outcomes of historical clinical trials and machine learning model predictions.

According to an embodiment of the system, the second risk profile data comprises planning data sets, wherein the planning data sets further comprises one or more of site recruitment plans, patient recruitment plans, a site forecast information, and a patient forecast information.

According to an embodiment of the system, the processor is further configured to assign a weighting to the risk categories based on a first pattern of outcomes of historical clinical trials and apply the weighting to factor values before generating the overall risk score.

According to an embodiment of the system, the processor is further configured to apply a different weighting to the risk categories at different time periods within the clinical trial.

According to an embodiment of the system, the processor is configured to modify the weighting of the risk elements based on a second pattern of outcomes of historical clinical trials.

According to an embodiment of the system, the processor is further configured to apply a different weighting to the risk elements at different time periods within the clinical trial.

According to an embodiment of the system, the processor is configured to display the overall risk score.

According to an embodiment of the system, the processor is further configured to display a chart showing correlation between the risk categories and the overall risk score.

According to an embodiment of the system, the level of monitoring comprises one or more of on-site monitoring with up to 100% Source Data Verification (SDV), remote monitoring with up to 100% SDV, no monitoring, and variations thereof.

According to an embodiment of the system, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

According to an embodiment of the system, the machine learning model comprises a neural network comprising a non-linear activation function configured to capture a non-linear association with the first risk profile data.

According to an embodiment of the system, the system is further configured for stratifying the type of monitoring into one of low, medium, and high risk categories using the overall risk score.

According to an embodiment of the system, the processor is further configured to perform one or more of a normalization, a standardization, and a stratification of the overall risk score.

According to an embodiment of the system, a high score of the overall risk score indicates a higher probability of monitoring and enabling proactive management throughout the clinical trial; and wherein a low score of the overall risk score indicates lower probability of monitoring and enabling proactive management throughout the clinical trial.

According to an embodiment of the system, the processor is further configured to predict the overall risk score using at least one of a logistic regression, a Support Vector Machine (SVM) regression, a convolutional neural network (CNN), a recurrent neural network (RNN), and a long short-term memory model (LSTM).

According to an embodiment, disclosed is a method comprising defining, one or more risk categories that influence risk associated with monitoring of a clinical trial, wherein the one or more risk categories comprise one or more risk elements; calculating, a first risk profile data of the one or more risk categories based on a risk factor and a weighting assigned to the one or more risk elements; generating, a machine learning model; training, the machine learning model with the first risk profile data; receiving, by the machine learning model, a second risk profile data based on the one or more risk categories and the one or more risk elements; analysing, by the machine learning model, the second risk profile data to identify a pattern based on the first risk profile data using a database; predicting, by the machine learning model and based on the pattern, an overall risk score for the clinical trial based on a predefined threshold value of the overall risk score; recommending, by the machine learning model based on the risk elements, one or more of a type of monitoring, a level of monitoring, and the overall risk score; updating the database with the second risk profile data; and wherein the machine learning model is a self-learning model comprising a feed-back layer that enables the machine learning model to learn from the second risk profile data and improve the prediction of the overall risk score and monitoring decisions to enable risk based monitoring (RBM) of the clinical trial.

According to an embodiment of the method, the risk categories comprise a global risk category, a local risk category, a site specific risk category, and a patient specific risk category.

According to an embodiment of the method, the risk elements are defined for the global risk category, the local risk category, the site specific risk category, and the patient specific risk category.

According to an embodiment of the method, the risk elements for the global risk category comprises global risk elements, the risk elements for the local risk category comprises local risk elements, the risk elements for the site specific risk category comprises site specific risk elements, and the risk elements for the patient specific risk category comprises patient specific risk elements.

According to an embodiment of the method, the global risk elements comprises therapeutic area, study phase, protocol complexity, an interventional, and an observational risk.

According to an embodiment of the method, the local risk elements comprises one or more of a geographic area, a socio economic profile, a site maturity profile, and a site experience profile.

According to an embodiment of the method, the site specific risk elements comprises one or more of a site feasibility, a prior history of a site, a site permanency, a site selection, and a site recruitment plan.

According to an embodiment of the method, the patient specific risk elements comprises one or more of a patient recruitment plan, a prior history of a patient, a patient selection criteria, and a patient recruitment forecast.

According to an embodiment of the method, the predefined threshold value of the overall risk score is initially set based on at least one of outcomes of historical clinical trials and machine learning model predictions.

According to an embodiment of the method, the second risk profile data comprises planning data sets, wherein the planning data sets further comprises one or more of site recruitment plans, patient recruitment plans, a site forecast information, and a patient forecast information.

According to an embodiment of the method, the method is further configured to assign a weighting to the risk categories based on a first pattern of outcomes of historical clinical trials and apply the weighting to factor values before generating the overall risk score.

According to an embodiment of the method, the method is further configured to apply a different weighting to the risk categories at different time periods within the clinical trial.

According to an embodiment of the method, the method is configured to modify the weighting of the risk elements based on a second pattern of outcomes of historical clinical trials.

According to an embodiment of the method, the method is further configured to apply a different weighting to the risk elements at different time periods within the clinical trial.

According to an embodiment of the method, the method is configured to display the overall risk score.

According to an embodiment of the method, the method is further configured to display a chart showing correlation between the risk categories and the overall risk score.

According to an embodiment of the method, the level of monitoring comprises one or more of an on-site monitoring with up to 100% Source Data Verification (SDV), a remote monitoring with up to 100% SDV, no monitoring, and variations thereof.

According to an embodiment of the method, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

According to an embodiment of the method, the machine learning model comprises a neural network comprising a non-linear activation function configured to capture a non-linear association with the first risk profile data.

According to an embodiment of the method, the method is further configured for stratifying the type of monitoring into one of low, medium, and high categories using the overall risk score.

According to an embodiment of the method, the method is further configured to perform a normalization, a standardization, and a stratification of the overall risk score.

According to an embodiment of the method, a high score of the overall risk score indicates a higher probability of monitoring the clinical trial; and wherein a low score of the overall risk score indicates a lower probability of monitoring the clinical trial.

According to an embodiment of the method, the method is further configured to predict the overall risk score using at least one of a logistic regression, a Support Vector Machine (SVM) regression, a convolutional neural network (CNN), a recurrent neural network (RNN), and a long short-term memory model (LSTM).

According to an embodiment, disclosed is a non-transitory computer-readable medium having stored thereon instructions executable by a computer system to perform operations comprising, defining, one or more risk categories that influence risk associated with monitoring of a clinical trial, wherein the one or more risk categories comprise one or more risk elements; calculating, a first risk profile data of the one or more risk categories based on a risk factor and a weighting assigned to the one or more risk elements; generating, a machine learning model; training, the machine learning model with the first risk profile data; receiving, by the machine learning model, a second risk profile data based on the one or more risk categories and the one or more risk elements; analysing, by the machine learning model, the second risk profile data to identify a pattern based on the first risk profile data using a database; predicting, by the machine learning model and based on the pattern, an overall risk score for the clinical trial based on a predefined threshold value of the overall risk score; recommending, by the machine learning model based on the one or more risk elements, a type of monitoring, a level of monitoring, and the overall risk score; updating the database with the second risk profile data; and wherein the machine learning model is a self-learning model comprising a feed-back layer that enables the machine learning model to learn from the second risk profile data and improve the prediction of the overall risk score and monitoring decisions to enable risk based monitoring (RBM) of the clinical trial.

According to an embodiment of the non-transitory computer-readable medium, the risk categories comprise one or more of a global risk category, a local risk category, a site specific risk category, and a patient specific risk category.

According to an embodiment of the non-transitory computer-readable medium, the risk elements are defined for each of the global risk category, the local risk category, the site specific risk category and the patient specific risk category.

According to an embodiment of the non-transitory computer-readable medium, the risk elements for the global risk category comprises global risk elements, the risk elements for the local risk category comprises local risk elements, the risk elements for the site specific risk category comprises site specific risk elements and the risk elements for the patient specific risk category comprises patient specific risk elements.

According to an embodiment of the non-transitory computer-readable medium, the global risk elements comprises one or more of a therapeutic area, a study phase, a protocol complexity, an interventional risk, and an observational risk.

According to an embodiment of the non-transitory computer-readable medium, the local risk elements comprises one or more of a geographic area, a socio economic profile, a site maturity profile, and a site experience profile.

According to an embodiment of the non-transitory computer-readable medium, the site specific risk elements comprises one or more of a site feasibility, a prior history of a site, a site permanency, a site selection, and a site recruitment plan.

According to an embodiment of the non-transitory computer-readable medium, the patient specific risk elements comprises one or more of a patient recruitment plan, a prior history of a patient, a patient selection criteria, and a patient recruitment forecast.

According to an embodiment of the non-transitory computer-readable medium, the predefined threshold value of the overall risk score is initially set based on outcomes of at least one of historical clinical trials and predicted by the machine learning model.

According to an embodiment of the non-transitory computer-readable medium, the second risk profile data comprises planning data sets, wherein the planning data sets further comprises one or more of site recruitment plans, patient recruitment plans, a site forecast information, and a patient forecast information.

According to an embodiment of the non-transitory computer-readable medium, the medium and the computer system are further configured to assign a weighting to the risk categories based on a first pattern of outcomes of historical clinical trials and apply the weighting to factor values before generating the overall risk score.

According to an embodiment of the non-transitory computer-readable medium, the medium and the computer system are further configured to apply a different weighting to the risk categories at different time periods within the clinical trial.

According to an embodiment of the non-transitory computer-readable medium, the instructions are configured to modify the weighting of the risk elements based on a second pattern of outcomes of historical clinical trials.

According to an embodiment of the non-transitory computer-readable medium, the instructions are further configured to apply a different weighting to the risk elements at different time periods within the clinical trial.

According to an embodiment of the non-transitory computer-readable medium, the computer system is configured to display the overall risk score.

According to an embodiment of the non-transitory computer-readable medium, the computer system is further configured to display a chart showing correlation between the risk categories and the overall risk score.

According to an embodiment of the non-transitory computer-readable medium, the level of monitoring comprises one or more of on-site monitoring with up to 100% Source Data Verification (SDV), remote monitoring with up to 100% SDV, no monitoring, and variations thereof.

According to an embodiment of the non-transitory computer-readable medium, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, a stacked neural network system.

According to an embodiment of the non-transitory computer-readable medium, the machine learning model comprises a neural network comprising a non-linear activation function to capture a non-linear association with the first risk profile data.

According to an embodiment of the non-transitory computer-readable medium, the instructions are further configured for stratifying the type of monitoring into one of low, medium, and high categories using the overall risk score.

According to an embodiment of the non-transitory computer-readable medium, the instructions are further configured to perform one or more of a normalization, a standardization, and a stratification of the overall risk score.

According to an embodiment of the non-transitory computer-readable medium, a high score of the overall risk score indicates a higher probability of monitoring the clinical trial; and wherein a low score of the overall risk score indicates a lower probability of monitoring the clinical trial.

According to an embodiment of the non-transitory computer-readable medium, the instructions are further configured to predict the overall risk score using at least one of a logistic regression, a Support Vector Machine (SVM) regression, a convolutional neural network (CNN), a recurrent neural network (RNN), and a long short-term memory model (LSTM).

According to an embodiment, disclosed is a method comprising, defining, one or more risk categories that influence risk associated with site monitoring of a clinical trial, wherein the one or more risk categories comprise one or more risk elements; calculating, a first site risk profile data of the one or more risk categories based on a risk factor and a weighting assigned to the one or more risk elements; generating, a machine learning model; training, the machine learning model with the first site risk profile data; receiving, by the machine learning model, a second site risk profile data based on the one or more risk categories and the one or more risk elements; analysing, by the machine learning model, the second site risk profile data to identify a pattern based on the first site risk profile data using a database; predicting, by the machine learning model and based on the pattern, a site risk score for the clinical trial based on a predefined threshold value of the site risk score; recommending, by the machine learning model based on the one or more risk elements, a type of monitoring, a level of monitoring, and the site risk score; updating the database with the second site risk profile data; and wherein the machine learning model is a self-learning model comprising a feed-back layer that enables the machine learning model to learn from the second site risk profile data and improve the prediction of the site risk score and monitoring decisions to enable risk based monitoring (RBM) of a site in the clinical trial.

According to an embodiment of the method, the risk categories comprise one or more of a local risk category, a site specific risk category and a patient specific risk category.

According to an embodiment of the method, the risk elements are defined for each of the local risk category, the site specific risk category and the patient specific risk category.

According to an embodiment of the method, the risk elements for the local risk category comprises local risk elements, the risk elements for the site specific risk category comprises site specific risk elements and the risk elements for the patient specific risk category comprises patient specific risk elements.

According to an embodiment of the method, the local risk elements comprises one or more of a geographic area, a socio economic profile, a site maturity profile, and a site experience profile.

According to an embodiment of the method, the site specific risk elements comprises one or more of a site feasibility, a prior history of a site, a site permanency, a site selection, and a site recruitment plan.

According to an embodiment of the method, the patient specific risk elements comprises one or more of a patient recruitment plan, a prior history of a patient, a patient selection criteria, and a patient recruitment forecast.

According to an embodiment of the method, the predefined threshold value of the site risk score is initially set based on outcomes of one or more of historical clinical trials and predicted by the machine learning model.

According to an embodiment of the method, the second site risk profile data comprises planning data sets, wherein the planning data sets further comprises one or more of site recruitment plans, patient recruitment plans, a site forecast information, and a patient forecast information.

According to an embodiment of the method, the method is further configured to assign a weighting to the risk categories based on a first pattern of outcomes of historical clinical trials and apply the weighting to factor values before generating the site risk score.

According to an embodiment of the method, the method is further configured to apply a different weighting to the risk categories at different time periods within the clinical trial.

According to an embodiment of the method, the method is configured to modify the weighting of the risk elements based on a second pattern of outcomes of historical clinical trials.

According to an embodiment of the method, the method is further configured to apply a different weighting to the risk elements at different time periods within the clinical trial.

According to an embodiment of the method, the method is configured to display the site risk score.

According to an embodiment of the method, the method is further configured to display a chart showing correlation between the risk categories and the site risk score.

According to an embodiment of the method, the level of monitoring comprises one or more of on-site monitoring with up to 100% Source Data Verification (SDV), a remote monitoring with up to 100% SDV, no monitoring, and variations thereof.

According to an embodiment of the method, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

According to an embodiment of the method, the machine learning model comprises a neural network comprising a non-linear activation function to capture a non-linear association with the first site risk profile data.

According to an embodiment of the method, the method is further configured for stratifying the type of monitoring into one of low, medium, and high categories using the site risk score.

According to an embodiment of the method, the method is further configured to perform one or more of a normalization, a standardization, and a stratification of the site risk score.

According to an embodiment of the method, higher scores of the site risk score indicates a higher probability of monitoring the clinical trial; and wherein a low score of the site risk score indicates a lower probability of monitoring the clinical trial.

According to an embodiment of the method, the method is further configured to predict the site risk score using at least one of a logistic regression, a Support Vector Machine (SVM) regression, a convolutional neural network (CNN), a recurrent neural network (RNN), and a long short-term memory model (LSTM).

According to an embodiment of the method, the training includes building a site-specific process review plan.

According to an embodiment of the method, the site monitoring includes reporting workflows with process reviews.

According to an embodiment of the method, the method further comprises reporting protocol deviations based on the site monitoring.

According to an embodiment, disclosed is a method comprising, defining, one or more risk categories that influence risk associated with patient monitoring in a clinical trial, wherein the one or more risk categories comprise one or more risk elements; calculating, a first patient risk profile data of the one or more risk categories based on a risk factor and a weighting assigned to the one or more risk elements; generating, a machine learning model; training, the machine learning model with the first patient risk profile data; receiving, by the machine learning model, a second patient risk profile data based on the one or more risk categories and the one or more risk elements; analysing, by the machine learning model, the second patient risk profile data to identify a pattern based on the first patient risk profile data using a database; predicting, by the machine learning model and based on the pattern, a patient risk score for the clinical trial based on a predefined threshold value of the patient risk score; recommending, by the machine learning model based on the one or more risk elements, a type of monitoring, a level of monitoring, and the patient risk score; updating the database with the second patient risk profile data; and wherein the machine learning model is a self-learning model comprising a feed-back layer that enables the machine learning model to learn from the second patient risk profile data and improve the prediction of the patient risk score and monitoring decisions to enable risk based monitoring (RBM) of patient in the clinical trial.

According to an embodiment of the method, the risk categories comprise one or more of a local risk category, a site specific risk category and a patient specific risk category.

According to an embodiment of the method, the risk elements are defined for each of the local risk category, the site specific risk category and the patient specific risk category.

According to an embodiment of the method, the risk elements for the local risk category comprises local risk elements, the risk elements for the site specific risk category comprises site specific risk elements and the risk elements for the patient specific risk category comprises patient specific risk elements.

According to an embodiment of the method, the local risk elements comprises one or more of a geographic area, a socio economic profile, a site maturity profile, and a site experience profile.

According to an embodiment of the method, the site specific risk elements comprises one or more of a site feasibility, a prior history of a site, a site permanency, a site selection, and a site recruitment plan.

According to an embodiment of the method, the patient specific risk elements comprises one or more of a patient recruitment plan, a prior history of a patient, a patient selection criteria, and a patient recruitment forecast.

According to an embodiment of the method, the predefined threshold value of the patient risk score is initially set based on outcomes of at least one of a historical clinical trials and predicted by the machine learning model.

According to an embodiment of the method, the second patient risk profile data comprises planning data sets, wherein the planning data sets further comprise one or more of site recruitment plans, patient recruitment plans, a site forecast information, and a patient forecast information.

According to an embodiment of the method, the method is further configured to assign a weighting to the risk categories based on a first pattern of outcomes of historical clinical trials and apply the weighting to factor values before generating the patient risk score.

According to an embodiment of the method, the method is further configured to apply a different weighting to the risk categories at different time periods within the clinical trial.

According to an embodiment of the method, the method is configured to modify the weighting of the risk elements based on a second pattern of outcomes of historical clinical trials.

According to an embodiment of the method, the method is further configured to apply different weighting to the risk elements at different time periods within the clinical trial.

According to an embodiment of the method, the method is configured to display the patient risk score.

According to an embodiment of the method, the method is further configured to display a chart showing correlation between the risk categories and the patient risk score.

According to an embodiment of the method, the level of monitoring comprises one or more of on-site monitoring with up to 100% Source Data Verification (SDV), remote monitoring with up to 100% SDV, no monitoring, and variations thereof.

According to an embodiment of the method, the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

According to an embodiment of the method, the machine learning model comprises a neural network comprising a non-linear activation function to capture a non-linear association with the first patient risk profile data.

According to an embodiment of the method, the method is further configured for stratifying the type of monitoring into one of low, medium, and high categories using the patient risk score.

According to an embodiment of the method, the method is further configured to perform one or more of a normalization, a standardization, and a stratification of the patient risk score.

According to an embodiment of the method, a high score of the patient risk score indicates a higher probability of monitoring the clinical trial; and wherein a low score of the patient risk score indicates a lower probability of monitoring the clinical trial.

According to an embodiment of the method, the method is further configured to predict the patient risk score using at least one of a logistic regression, a Support Vector Machine (SVM) regression, a convolutional neural network (CNN), a recurrent neural network (RNN), and a long short-term memory model (LSTM).

According to an embodiment of the method, the training includes building a patient-specific process review plan.

According to an embodiment of the method, the patient monitoring includes reporting workflows with process reviews.

According to an embodiment of the method, further comprising reporting protocol deviations based on the patient monitoring.

According to an embodiment, disclosed is a system comprising a processor storing instructions in a non-transitory memory that, when executed, cause the processor to receive, one of risk categories and risk elements; calculate, a first risk profile data based on a risk factor and a weighting; receive, a second risk profile data from an artificial intelligence engine; analyse, second risk profile data to identify a pattern based on the first risk profile data; predict, an overall risk score for clinical trial based on the pattern; recommend, one or more of a type of monitoring and a level of monitoring based on the overall risk score; update a database with the second risk profile data; and wherein the machine learning model is configured for continual learning from the second risk profile data to improve prediction and monitoring decisions.

According to an embodiment, disclosed is a system comprising a processor storing instructions in a non-transitory memory that, when executed, cause the processor to send one of a one or more risk categories and risk elements to a clinical trial analysis and recommendation system via a client system; send data, wherein the data comprises one or more of a patient data, a site data, a drug molecules, a disease information, and a trial protocol, via the client system; receive, a risk score for a clinical trial based on the risk categories and an overall risk score for the clinical trial; send, a predefined threshold value for the risk score for the clinical trial; receive, a second overall risk score for the clinical trial based on the predefined threshold value of the overall risk score; and receive a recommendation, by the machine learning model based on the risk elements, one or more of a type of monitoring, a level of monitoring, and the overall risk score.

The descriptions of the one or more embodiments are for purposes of illustration but are not exhaustive or limiting to the embodiments described herein. Many modifications and variations will be apparent to those of ordinary skill in the art without departing from the scope and spirit of the described embodiments. The terminology used herein best explains the principles of the embodiments, the practical application and/or technical improvement over technologies found in the marketplace, and/or to enable others of ordinary skill in the art to understand the embodiments described herein.

### INCORPORATION BY REFERENCE

All references, including granted patents and patent application publications, referred herein are incorporated herein by reference in their entirety:

U.S. Pat. No. 8,706,537B1 titled “Remote clinical study site monitoring and data quality scoring”;

US20240054419A1 titled “Site monitoring activity review tool (art)”

1-124. (canceled)

125. A system comprising:
a processor storing instructions in a non-transitory memory that, when executed, cause the processor to:
define, one or more risk categories that influence risk associated with monitoring of a clinical trial, wherein the risk categories comprise one or more risk elements:
calculate, a first risk profile data of the risk categories based on a risk factor and a weighting assigned to the risk elements:
train, a machine learning model with the first risk profile data;
receive, by the machine learning model, a second risk profile data based on the risk categories and the risk elements:
analyse, by the machine learning model, the second risk profile data to identify a pattern based on the first risk profile data using a database;
apply at least one of a normalization, standardization, or transformation fiction to the second risk profile data:
predict, by the machine learning model and based on the pattern, an overall risk score for the clinical trial based on a predefined threshold value of the overall risk score;
recommend, by the machine learning model based on the risk elements, one or more of a type of monitoring, a level of monitoring, and the overall risk score; and
update the database with the second risk profile data;
generate, a graphical user interface (GUI) that visually displays the overall risk score along with a correlation chart between the risk categories and the overall risk score;
stratify the type of monitoring into one of low, medium, and high categories using the overall risk score; and
wherein the machine learning model is a self-learning model comprising a feed-back layer that enables the machine leaning model to learn from the second risk profile data and improve a prediction of the overall risk score and monitoring decisions to enable risk based monitoring (RBM) of the clinical trial.

126. The system of claim 125, wherein the risk categories comprise global risk category, local risk category, site specific risk category, and patient specific risk category.

127. The system of claim 126, wherein the risk elements are defined for each of the global risk category, the local risk category, the site specific risk category and the patient specific risk category, and wherein the risk elements for the global risk category comprises global risk elements, the risk elements for the local risk category comprises local risk elements, the risk elements for the site specific risk category comprises site specific risk elements, and the risk elements for the patient specific risk category comprises patient specific risk elements.

128. The system of claim 127, wherein the global risk elements comprises one or more of a therapeutic area, a study phase, a protocol complexity, an interventional risk, and an observational risk.

129. The system of claim 127, wherein the local risk elements comprises one or more of a geographic area, a socio economic profile, a site maturity profile, and a site experience profile.

130. The system of claim 127, wherein the site specific risk elements comprises one or more of a site feasibility, a prior history of a site, a site permanency, a site selection, and a site recruitment plan.

131. The system of claim 127, wherein the patient specific risk elements comprises one or more of a patient recruitment plans, a prior history of a patient, a patient selection criteria, and a patient recruitment forecast.

132. The system of claim 125, wherein the predefined threshold value of the overall risk score is initially set based on at least one of outcomes of historical clinical trials and predicted by the machine learning model.

133. The system of claim 125, wherein the second risk profile data comprises planning data sets, wherein the planning data sets further comprises one or more of site recruitment plans, patient recruitment plans, a site forecast information, and a patient forecast information.

134. The system of claim 125, wherein the processor is further configured to assign a weighting to the risk categories based on a first pattern of outcomes of historical clinical trials and apply the weighting to factor values before generating the overall risk score, and wherein the weighting is different at different time periods within the clinical trial.

135. The system of claim 125, wherein the processor is further configured to modify the weighting of the risk elements based on a second pattern of outcomes of historical clinical trials, wherein the weighting is different at different time periods within the clinical trial.

136. The system of claim 125, wherein the level of monitoring comprises one or more of on-site monitoring with up to 100% Source Data Verification (SDV), remote monitoring with up to 100% SDV, no monitoring, and variations thereof.

137. The system of claim 125, wherein the machine learning model comprises at least one of a convolution neural network, a recurrent neural network, a deep neural network, and a stacked neural network, rules-based system, a decision tree-based system, a logical condition-based system, a causal probabilistic network system, a Bayesian network system, a support vector machine, a neural network system, and a stacked neural network system.

138. The system of claim 125, wherein the machine learning model comprises a neural network comprising a non-linear activation function configured to capture a non-linear association with the first risk profile data.

139. The system of claim 125, wherein the machine learning model comprises an explainable AI algorithm, wherein the explainable AI algorithm is configured to provide reasoning for the prediction of the overall risk score and the monitoring decisions; and build trust parameters for users of the model.

140. The system of claim 125, wherein the processor is further configured to predict the overall risk score using at least one of a logistic regression, a Support Vector Machine (SVM) regression, a convolutional neural network (CNN), a recurrent neural network (RNN), and a long short-term memory model (LSTM).

141. A method comprising:
defining, one or more risk categories that influence risk associated with monitoring of a clinical trial, wherein the one or more risk categories comprise one or more risk elements:
calculating, a first risk profile data of the one or more risk categories based on a risk factor and a weighting assigned to the one or more risk elements:
training, a machine learning model with the first risk profile data;
receiving, by the machine learning model, a second risk profile data based on the one or more risk categories and the one or more risk elements;
analysing, by the machine learning model, the second risk profile data to identify a pattern based on the first risk profile data using a database;
applying at least one of a normalization, standardization, or transformation function to the second risk profile data;
predicting, by the machine learning model and based on the pattern, an overall risk score for the clinical trial based on a predefined threshold value of the overall risk score;
recommending, by the machine learning model based on the risk elements, one or more of a type of monitoring, a level of monitoring, and the overall risk score; and
updating the database with the second risk profile data;
generating, a graphical user interface (GUI) that visually displays the overall risk score along with a correlation chart between the risk categories and the overall risk score:
stratifying the type of monitoring into one of low, medium, and high categories using the overall risk score; and
wherein the machine learning model is a self-learning model comprising a feed-back layer that enables the machine learning model to learn from the second risk profile data and improve a prediction of the overall risk score and monitoring decisions to enable risk based monitoring (RBM) of the clinical trial.

142. The method of claim 141, wherein the risk categories comprise a global risk category, a local risk category, a site specific risk category, and a patient specific risk category.

143. (canceled)

144. A non-transitory computer-readable medium having stored thereon instructions executable by a computer system to perform operations comprising:
defining, one or more risk categories that influence risk associated with monitoring of a clinical trial, wherein the one or more risk categories comprise one or more risk elements;
calculating, a first risk profile data of the one or more risk categories based on a risk factor and a weighting assigned to the one or more risk elements:
training, a machine learning model with the first risk profile data;
receiving, by the machine learning model, a second risk profile data based on the one or more risk categories and the one or more risk elements;
analysing, by the machine learning model, the second risk profile data to identify a pattern based on the first risk profile data using a database;
applying at least one of a normalization, standardization, or transformation function to the second risk profile data;
predicting, by the machine learning model and based on the pattern, an overall risk score for the clinical trial based on a predefined threshold value of the overall risk score;
recommending, by the machine learning model based on the one or more risk elements, a type of monitoring, a level of monitoring, and the overall risk score; and
updating the database with the second risk profile data;
generating, a graphical user interface (GUI) that visually displays the overall risk score along with a correlation chart between the risk categories and the overall risk score;
stratifying the type of monitoring into one of low, medium, and high categories using the overall risk score; and
wherein the machine learning model is a self-learning model comprising a feed-back layer that enables the machine learning model to learn from the second risk profile data and improve a prediction of the overall risk score and monitoring decisions to enable risk based monitoring (RBM) of the clinical trial.
